(19) United States
(12) Patent Application Publication
(10) Pub. No.: US 2025/0295369 A1
(43) Pub. Date: Sep. 25, 2025

(54) MEDICAL IMAGE PROCESSING APPARATUS AND X-RAY DIAGNOSTIC APPARATUS

(71) Applicant: Canon Medical Systems Corporation, Otawara-shi (JP)

(72) Inventor: Takuya SAKAGUCHI, Utsunomiya (JP)

(73) Assignee: Canon Medical Systems Corporation, Otawara-shi (JP)

(21) Appl. No.: 19/083,619

(22) Filed: Mar. 19, 2025

(30) Foreign Application Priority Data
Mar. 19, 2024 (JP) 2024-043857

(51) Int. Cl. A61B 6/00; A61B 6/46; A61B 6/50

(52) U.S. Cl. CPC A61B 6/484; A61B 6/461; A61B 6/503; A61B 6/504

(57) ABSTRACT

According to one embodiment, a medical image processing apparatus acquires a zeroth contrast-enhanced image, a first contrast-enhanced image, and a second contrast-enhanced image. The medical image processing apparatus calculates a first blood flow ratio between a zeroth blood flow rate in a myocardium in the reference state and a first blood flow rate in the acetylcholine stress state based on the zeroth contrast-enhanced image and the first contrast-enhanced image. The medical image processing apparatus calculates a second blood flow ratio between the zeroth blood flow rate and a second blood flow rate in the myocardium in the adenosine stress state based on the zeroth contrast-enhanced image and the second contrast-enhanced image.

### CROSS-REFERENCE TO RELATED APPLICATION

This application is based upon and claims the benefit of priority from the prior Japanese Patent Application No. 2024-043857, filed Mar. 19, 2024, the entire contents of which are incorporated herein by reference.

### FIELD

Embodiments described herein relate generally to a medical image processing apparatus and an X-ray diagnostic apparatus.

### BACKGROUND

In the field of ischemia in the circulatory organ, there is a trend toward solving INOCA (Ischemia with No-Obstructive Coronary Artery disease) with a functional abnormality, that is, the blood is difficult to flow in the myocardium despite no functional abnormality such as the occlusion or coarctation of the cardiac blood vessel. There are two types of main factors for INOCA, namely, microvascular spasm and no microvascular dilation. It is expected to provide correct medical treatment by properly discriminating these causes. In addition, in discriminating the causes, a method as a gold standard uses evaluation such as IMR (microcirculatory resistance) using a sensor wire in invasive examination or CFR (coronary flow reverse) or lactic acid because noninvasive examination such as MRI (magnetic resonance imaging), PET (positron emission tomography), or transthoracic echocardiography is low in sensitivity.

In addition to the above description, invasive examination includes the first examination associated with coronary spastic angina attributed to one of the two types of main causes and the second examination associated with microvascular angina attributed to the other cause. Invasive examination properly discriminates the cause of INOCA by using these two types of examination. The order of the first examination and the second examination is reversed depending on the country or the like and is not limited to this.

First of all, the first examination determines whether the examination result is positive or not from symptoms, electrocardiogram changes, and lactate levels upon performing an acetylcholine stress test as a provocative test for coronary artery spasm associated with vasospastic angina. If the first examination determines that the examination result is positive, the patient is subjected to medical treatment for vasospastic angina as one of the causes.

If the first examination determines that the examination result is negative, the second examination associated with the other cause is performed. In the second examination, adenosine stress examination associated with microvascular angina is conducted to determine from CFR and IMR whether the examination result is positive. If the second examination result is positive, the patient is subjected to medical treatment for microvascular angina as the other cause.

According to the studies conducted by the present inventor, however, there is room for improvement in invasive examination. For example, the first examination has room for improvement in that although determination is based on symptoms, electrocardiogram changes, and lactate levels, the blood flow representing the actual degree of ischemia cannot be quantitatively measured. In addition, for example, the second examination has room for improvement in that the examination takes time and effort and cost because of the use of a sensor wire, all coronary artery branches cannot be evaluated, and there is a risk in inserting a sensor wire.

Accordingly, in examination for INOCA, it is preferable to quantitatively measure the blood flow without using any sensor wire.

### BRIEF DESCRIPTION OF THE DRAWINGS

FIG. 1 is a block diagram showing an example of a medical image processing apparatus and its peripheral arrangement according to the first embodiment.

FIG. 2 is a flowchart for explaining an operation according to the first embodiment.

FIG. 3 is a view showing an example of contrast-enhanced images according to the first embodiment.

FIG. 4 is a schematic view showing a display example according to the first embodiment.

FIG. 5 is a schematic view for explaining a display example of ratio images according to the second embodiment.

FIG. 6 is a schematic view for explaining a display example of ratio images according to a modification of the second embodiment.

FIG. 7 is a schematic view showing a display example of graphs according to the third embodiment.

FIG. 8 is a schematic view showing a display example of graphs according to the fourth embodiment.

FIG. 9 is a schematic view showing a display example of graphs according to a modification of the fourth embodiment.

FIG. 10 is a schematic view showing a display example of graphs according to a modification of the fourth embodiment.

FIG. 11 is a schematic view showing a display example of graphs according to a modification of the fourth embodiment.

FIG. 12 is a schematic view showing a display example of graphs according to a modification of the fourth embodiment.

FIG. 13 is a block diagram showing an example of a medical image processing apparatus and its peripheral arrangement according to the fifth embodiment.

FIG. 14 is a flowchart for explaining an operation according to the fifth embodiment.

FIG. 15 is a schematic view showing a display example according to the sixth embodiment.

FIG. 16 is a block diagram showing an example of the arrangement of an X-ray diagnostic apparatus according to the seventh embodiment.

### DETAILED DESCRIPTION

In general, according to one embodiment, a A medical image processing apparatus includes processing circuitry. The processing circuitry is configured to acquire a zeroth contrast-enhanced image when a heart of a subject is in a reference state, a first contrast-enhanced image when the heart is in an acetylcholine stress state, and a second contrast-enhanced image when the heart is in an adenosine stress state. The processing circuitry is configured to calculate a first blood flow ratio representing a ratio between a zeroth blood flow rate in a myocardium in the reference state and a first blood flow rate in the acetylcholine stress state based on the zeroth contrast-enhanced image and the first contrast-enhanced image. The processing circuitry is configured to calculate a second blood flow ratio representing a ratio between the zeroth blood flow rate and a second blood flow rate in the myocardium in the adenosine stress state based on the zeroth contrast-enhanced image and the second contrast-enhanced image. The processing circuitry is configured to cause a display to display the first blood flow ratio and the second blood flow ratio side by side.

A medical image processing apparatus and an X-ray diagnostic apparatus according to each embodiment will be described below with reference to the accompanying drawings. In the following description, the same reference numerals denote constituent elements having almost the same functions and arrangements, and a repetitive explanation will be omitted. In addition, in the following description, a description of information that is not always necessary to understand the measurement or display of blood flow ratios will be omitted as needed.

### First Embodiment

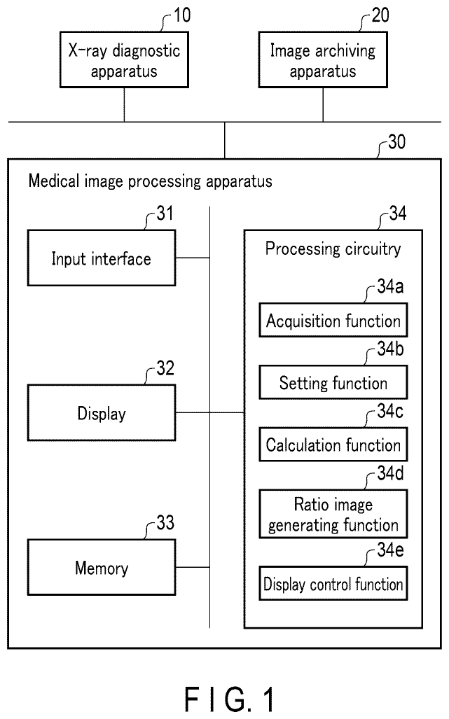

FIG. 1 is a block diagram showing an example of a medical image processing apparatus and its peripheral arrangement according to the first embodiment. An X-ray diagnostic apparatus 10, an image archiving apparatus 20, and a medical image processing apparatus 30 are communicably connected to each other via a wired or wireless network. The network is, for example, a LAN (Local Area Network). Note that as long as security is secured by VPN (Virtual Private Network) or the like, the line to be connected is not limited to a LAN. At this time, the network may be, for example, a public communication line such as the Internet.

The X-ray diagnostic apparatus 10 acquires the data of a projected image of a subject by radiography of the subject. The X-ray diagnostic apparatus 10 also acquires the data of a contrast-enhanced image associated with the cardiac region of the subject having a contrast medium injected in the cardiac region by radiography of the subject. Note that the data of a projection image and a contrast-enhanced image each accompany radiography conditions. The radiography conditions include, for example, a radiography target region, a tube voltage, a tube current, the product of the tube current (mA) and the irradiation time(s) (to be referred to as a tube current time product (mAs) hereinafter), the body position (posture) of a subject at the time of radiography, and the injection amount/injection rate of a contrast medium. Scan conditions and radiography conditions correspond to imaging conditions. Volume data, contrast-enhanced image data, and projection image data correspond to medical image data. A body position is the posture of a subject at the time of radiography of the subject. For example, body positions in scan conditions include a supine position, a both upper limbs raised position, a dorsal position, a lateral position, and a prone position. In addition, body positions in radiography conditions include, for example, a dorsal position, a lateral position, and a both limbs descent position. Note that the body position of a subject may not be included in imaging conditions.

The image archiving apparatus 20 is an apparatus that archives the medical image data acquired by the X-ray diagnostic apparatus 10. The image archiving apparatus 20 acquires medical image data from the X-ray diagnostic apparatus 10 via a network and causes a memory provided inside or outside the apparatus to store the acquired contrast-enhanced image data. For example, the image archiving apparatus 20 is implemented by computer equipment such as a server apparatus.

The medical image processing apparatus 30 acquires medical image data from the X-ray diagnostic apparatus 10 or the image archiving apparatus 20 via the network and executes various types of processing using the acquired medical image data. The medical image processing apparatus 30 is implemented by, for example, computer equipment such as a workstation. Note that the X-ray diagnostic apparatus 10, the image archiving apparatus 20, and the medical image processing apparatus 30 can be installed in arbitrary places as long as they can be connected via a network. For example, the medical image processing apparatus 30 may be installed in a facility, hospital, or the like different from the place where the X-ray diagnostic apparatus 10 is installed. In addition, the medical image processing apparatus 30 may be mounted in the X-ray diagnostic apparatus 10.

The medical image processing apparatus 30 includes an input interface 31, a display 32, a memory 33, and a processing circuitry 34.

The input interface 31 accepts various types of input operations from the operator, converts the accepted input operations into electrical signals, and outputs the converted electrical signals to the processing circuitry 34. For example, the input interface 31 is implemented by a touch pad that performs an input operation when the operator touches a mouse, a keyboard, a trackball, a switch, a button, a joystick, and an operation surface, a touch screen obtained by integrating a display screen and a touch pad, a non-contact input circuit using an optical sensor, a speech input circuit, or the like. Note that the input interface 31 may be implemented by the medical image processing apparatus 30, a wirelessly communicable tablet terminal, and the like. The input interface 31 is not limited to only a device including a physical operation component such as a mouse or keyboard. For example, another example of the input interface 31 is an electrical signal processing circuitry that accepts an electrical signal corresponding to an input operation from an external input device provided separately from the medical image processing apparatus 30 and outputs this electrical signal to the processing circuitry 34. The input interface 31 is an example of an input unit.

The display 32 is implemented by a display main body that displays various types of information such as medical images, an internal circuit that supplies a display signal to the display main body, and a peripheral circuit, such as a connector or cable, that connects the display main body to the internal circuit. The internal circuit generates display data by superimposing supplementary information such as subject information and projection data generation conditions on the image data supplied from the processing circuitry 34, performs D/A conversion and TV format conversion with respect to the obtained display data, and displays the resultant data on the display main body. For example, the display 32 outputs the medical image acquired and generated by the processing circuitry 34, a GUI (Graphical User Interface) for accepting various types of operations from the operator, and the like. For example, the display 32 is a liquid crystal display or CRT (Cathode Ray Tube) display. The display 32 is an example of a display unit. The display 32 may be of a desktop type or may be implemented by a tablet terminal or the like that is wirelessly communicable with the main body of the medical image processing apparatus 30. The display 32 is an example of a display unit.

The memory 33 is a memory device such as a ROM (Read Only Memory) that stores various types of information, RAM (Random Access Memory), HDD (Hard Disk Drive), SSD (Solid State Drive), or integrated circuit memory device. The memory 33 may be a drive device that reads and writes various types of information between itself and portable memory devices such as a CD-ROM drive, a DVD drive, and a flash memory. Note that the memory 33 need not always be implemented by a single memory device. For example, the memory 33 may be implemented by a plurality of memory devices. In addition, the memory 33 may be installed in another computer connected to the medical image processing apparatus 30 via a network.

The memory 33 stores the medical image data acquired from the X-ray diagnostic apparatus 10 or the image archiving apparatus 20. Examples of medical image data are a zeroth contrast-enhanced image when the heart of a subject having ischemia with no-obstructive coronary artery disease is in a reference state, a first contrast-enhanced image when the heart is in an acetylcholine stress state, and a second contrast-enhanced image when the heart is in an adenosine stress state. The zeroth contrast-enhanced image, the first contrast-enhanced image, and the second contrast-enhanced image are medical images obtained by radiography under almost the same radiography conditions except for the states of the heart. The memory 33 stores various types of data before, during, and after processing using various types of functions of the processing circuitry 34. Various types of data include, for example, index data such as a blood flow rate or blood flow ratio and image data such as a ratio image based on index data. The memory 33 stores programs for causing the computer to implement various types of functions of the processing circuitry 34 mounted in the medical image processing apparatus 30. These programs may be stored in advance in the memory 33. In addition, for example, the programs may be stored in a non-transitory computer readable storage medium and distributed. The programs may then be read out from the non-transitory computer readable storage medium and installed in the memory 33. The memory 33 is an example of a storage unit.

The processing circuitry 34 controls the overall operation of the medical image processing apparatus 30 in accordance with electrical signals of input operations output from the input interface 31. For example, the processing circuitry 34 includes, as hardware resources, a processor such as a CPU, MPU, or GPU (Graphic Processing Unit) and a memory such as a ROM and a RAM. The processing circuitry 34 is a processor that implements an acquisition function 34a, a setting function 34b, a calculation function 34c, a ratio image generating function 34d, and a display control function 34e corresponding to the programs by invoking and executing the programs in the memory 33. According to the above description with reference to FIG. 1, the single processing circuitry 34 implements the acquisition function 34a, the setting function 34b, the calculation function 34c, the ratio image generating function 34d, and the display control function 34e. However, a processing circuitry may be implemented by combining a plurality of independent processors, and each processor may implement a corresponding one of the functions by executing the program. The acquisition function 34a, the setting function 34b, the calculation function 34c, the ratio image generating function 34d, and the display control function 34e may be respectively referred to as an acquisition circuit, a setting circuit, a calculation circuit, a ratio image generating circuit, and a display control circuit or implemented as individual hardware circuits. In addition, the various functions of the processing circuitry 34 may be mounted in, for example, the processing circuitry of the X-ray diagnostic apparatus 10. In this case, the medical image processing apparatus 30 is incorporated in the X-ray diagnostic apparatus 10.

The acquisition function 34a acquires the zeroth contrast-enhanced image when the heart of the subject is in the reference state, the first contrast-enhanced image when the heart is in the acetylcholine stress state, and the second contrast-enhanced image when the heart is in the adenosine stress state. In this case, the reference state is a state serving as a reference when a blood flow ratio is calculated, more specifically, a state without administration of a medical agent that causes spasm or dilation of the cardiac blood vessel. Accordingly, the reference state may be renamed as a normal state, standard state, or a no-stress state. The acetylcholine stress state is a state with administration of a medical agent (acetylcholine) that causes spasm of the cardiac blood vessel. The adenosine stress state is a state with administration of a medical agent (adenosine) that causes dilation of the cardiac blood vessel. The acquisition destination of each contrast-enhanced image acquired by the acquisition function 34a is, for example, the image archiving apparatus 20. The acquisition function 34a saves each acquired contrast-enhanced image in the memory 33. The acquisition function 34a is an example of an acquisition unit.

The setting function 34b sets a region of interest common to the zeroth contrast-enhanced image, the first contrast-enhanced image, and the second contrast-enhanced image. For example, the setting function 34b may manually set a region of interest for one of the zeroth to second contrast-enhanced images and automatically set regions of interest for the remaining two contrast-enhanced images. More specifically, for example, the setting function 34b may set a region of interest for the zeroth contrast-enhanced image in accordance with an operation by the operator and set regions of interest at the same positions in the first contrast-enhanced image and the second contrast-enhanced image in conjunction with the position of the region of interest in the zeroth contrast-enhanced image. The setting function 34b is an example of a setting unit.

The calculation function 34c calculates the first blood flow ratio representing the ratio between the zeroth blood flow rate in the myocardium in the reference state and the first blood flow rate in the myocardium in the acetylcholine stress state based on the zeroth contrast-enhanced image and the first contrast-enhanced image. For example, the calculation function 34c may calculate the first blood flow ratio in each of the regions of interest in the zeroth contrast-enhanced image and the first contrast-enhanced image. The first blood flow ratio in the region of interest may be calculated as, for example, the maximum value or average value of the absolute values of blood flow ratios in the respective pixels in the region of interest. In addition, for example, the calculation function 34c may calculate the first blood flow ratio for each corresponding pixel in the zeroth contrast-enhanced image and the first contrast-enhanced image. In addition, for example, the calculation function 34c may calculate the first blood flow ratio by dividing the pixel values of corresponding pixels in the myocardial region in the zeroth contrast-enhanced image and the myocardial region in the first contrast-enhanced image.

The calculation function 34c calculates the second blood flow ratio representing the ratio between the zeroth blood flow rate and the second blood flow rate in the myocardium in the adenosine stress state based on the zeroth contrast-enhanced image and the second contrast-enhanced image. For example, the calculation function 34c may calculate the second blood flow ratio in each of the regions of interest in the zeroth contrast-enhanced image and the second contrast-enhanced image. The second blood flow ratio in the region of interest may be calculated as the maximum value or average value of the absolute values of blood flow ratios for the respective pixels in the region of interest. In addition, for example, the calculation function 34c may calculate the second blood flow ratio for each corresponding pixel in the zeroth contrast-enhanced image and the second contrast-enhanced image. In addition, for example, the calculation function 34c may calculate the second blood flow ratio by dividing the pixel values of corresponding pixels in the myocardial region in the zeroth contrast-enhanced image and the myocardial region in the second contrast-enhanced image. The calculation function 34c is an example of the first calculation unit and the second calculation unit.

The ratio image generating function 34d generates the first ratio image having a pixel value corresponding to the first blood flow ratio calculated for each pixel and the second ratio image having a pixel value corresponding to the second blood flow ratio calculated for each pixel. The ratio image generating function 34d is an example of a ratio image generating unit.

The display control function 34e controls the display so as to display a desired screen. For example, the display control function 34e causes the display 32 to display the first blood flow ratio and the second blood flow ratio side by side. In addition, for example, the display control function 34e may cause the display 32 to display the first blood flow ratio and the second blood flow ratio at least in a numerical value form or an image form. For example, in the case of the numerical value form, the display control function 34e may cause the display 32 to display the first blood flow ratio in the first display mode if the first blood flow ratio in the region of interest is equal to or less than the first threshold and to display the second blood flow ratio in the second display mode if the second blood flow ratio in the region of interest is equal to or less than the second threshold. In this case, the first display mode may be, for example, a mode of performing color display or alert display indicating abnormality in the first blood flow ratio. The second display mode may be, for example, a mode of performing color display or alert display indicating abnormality in the second blood flow ratio. In addition, in the case of the image form, the display control function 34e may cause the display 32 to perform color display of the first ratio image and the second ratio image by assigning colors to pixel values. The display control function 34e may also cause the display 32 to display at least the first blood flow rate and the second blood flow rate of the zeroth blood flow rate, the first blood flow rate, and the second blood flow rate. The display control function 34e is an example of a display control unit.

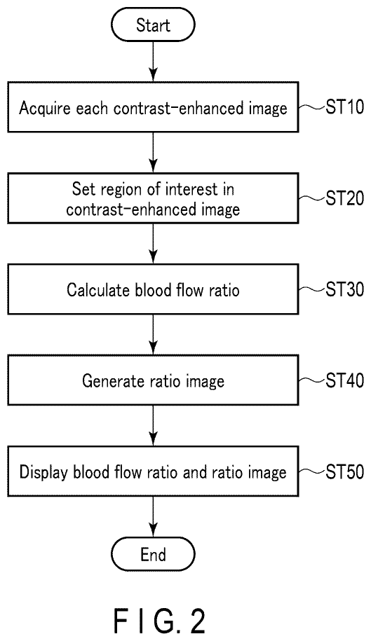

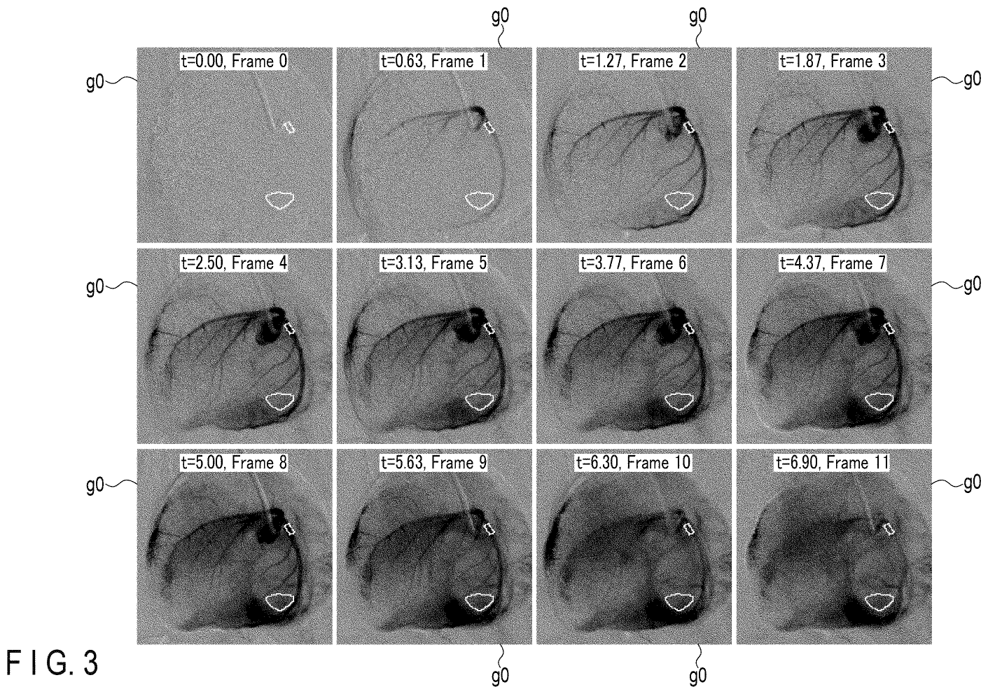

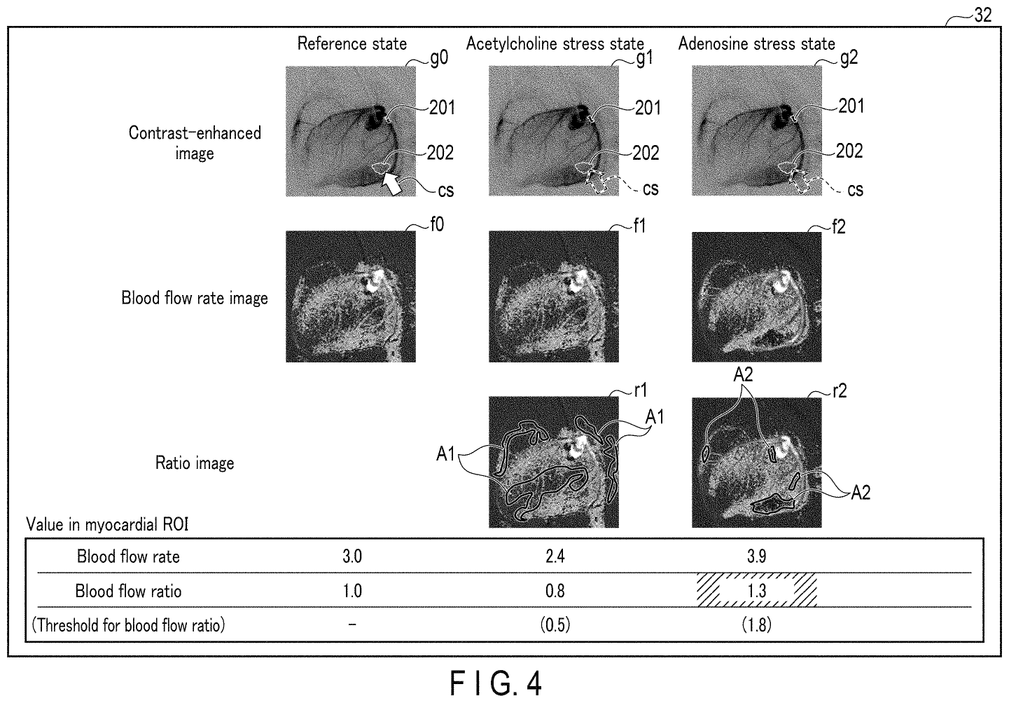

The operation of the medical image processing apparatus having the above arrangement will be described next with reference to the flowchart of FIG. 2 and the schematic views of FIGS. 3 and 4. Assume that the image archiving apparatus 20 saves, in advance, the zeroth contrast-enhanced image, the first contrast-enhanced image, and the second contrast-enhanced image acquired by the X-ray diagnostic apparatus 10. The zeroth contrast-enhanced image is an X-ray image when the heart of the subject having ischemia with no-obstructive coronary artery disease is in the reference state. The first contrast-enhanced image is an X-ray image when the heart is in the acetylcholine stress state. The second contrast-enhanced image is an X-ray image when the heart is in the adenosine stress state.

### (Step ST10)

As shown in FIG. 2, the processing circuitry 34 of the medical image processing apparatus 30 acquires the zeroth contrast-enhanced image, the first contrast-enhanced image, and the second contrast-enhanced image of the subject from the image archiving apparatus 20 and saves each contrast-enhanced image in the memory 33. As shown in FIG. 3, a zeroth contrast-enhanced image g0 is a moving image having a plurality of frames in chronological order and represents, in chronological order, how the contrast medium injected through a catheter flows out to the myocardial region through the coronary artery and capillaries. The same applies to the first contrast-enhanced image and the second contrast-enhanced image.

### (Step ST20)

As indicated by the upper row of FIG. 4, the processing circuitry 34 causes the display 32 to display the zeroth contrast-enhanced image g0, a first contrast-enhanced image g1, and a second contrast-enhanced image g2 in accordance with an operation by the operator. In addition, the processing circuitry 34 sets a region of interest (ROI) common to the zeroth contrast-enhanced image, the first contrast-enhanced image, and the second contrast-enhanced image in accordance with an operation by the operator. For example, the processing circuitry 34 sets an artery ROI 201 in part of the coronary artery in the zeroth contrast-enhanced image g0 in accordance with the operation of a cursor cs and also sets a myocardial ROI 202 in part of the myocardial region in the zeroth contrast-enhanced image g0. In addition, the processing circuitry 34 respectively sets the artery ROIs 201 at the same positions in the first contrast-enhanced image g1 and the second contrast-enhanced image g2 in conjunction with the position of the artery ROI 201 in the zeroth contrast-enhanced image g0. Likewise, the processing circuitry 34 respectively sets the myocardial ROIs 202 at the same positions in the first contrast-enhanced image g1 and the second contrast-enhanced image g2 in conjunction with the position of the myocardial ROI 202 in the zeroth contrast-enhanced image g0.

### (Step ST30)

The processing circuitry 34 calculates the first blood flow ratio representing the ratio between the zeroth blood flow rate in the myocardium in the reference state and the first blood flow rate in the myocardium in the acetylcholine stress state based on the zeroth contrast-enhanced image g0 and the first contrast-enhanced image g1. For example, the processing circuitry 34 calculates the first blood flow ratio in each of the myocardial ROIs 202 in the zeroth contrast-enhanced image g0 and the first contrast-enhanced image g1. Note that the first blood flow ratio in the myocardium may be obtained by calculating the zeroth blood flow rate and the first blood flow rate and dividing the first blood flow rate by the zeroth blood flow rate.

For example, let La be the vascular thickness of the subject along an X-ray path, Lm be the myocardial thickness of the subject along the X-ray path, Im(t) be the luminance of the myocardium in the zeroth contrast-enhanced image g0 at time t elapsed from the start of injection of a contrast medium, and Ia(t) be the luminance of the blood vessel in the zeroth contrast-enhanced image g0 at time τ (0≤τ≤t). At this time, the processing circuitry 34 calculates a zeroth blood flow rate K1 in the myocardium in the reference state based on equation (1).

$${K}_{1} = \frac{{L}_{a}}{{L}_{m}} \frac{{I}_{m} ( t )}{{\int }_{0}^{t} {I}_{a} ( τ ) d τ} \qquad ( 1 )$$

Likewise, let Im(t)ACh be the luminance of the myocardium in the first contrast-enhanced image g1 at time t and Ia(τ) be the luminance of the blood vessel in the first contrast-enhanced image g1 at time τ. At this time, the processing circuitry 34 calculates a first blood flow rate K1_ACh in the myocardium in the acetylcholine stress state based on equation (2).

$${K}_{1 \_ ACh} = \frac{{L}_{a}}{{L}_{m}} \frac{{I}_{m} ( t ) ACh}{{\int }_{0}^{t} {I}_{a} ( τ ) d τ} \qquad ( 2 )$$

Subsequently, the processing circuitry 34 calculates a first blood flow ratio (K1_ACh/K1) in the myocardium by dividing the calculated first blood flow rate K1_ACh by the calculated zeroth blood flow rate K1.

Likewise, let Im(t)ATP be the luminance of the myocardium in the second contrast-enhanced image g2 at time t, and Ia(τ) be the luminance of the myocardium in the second contrast-enhanced image g2 at time (τ). The processing circuitry 34 calculates a second blood flow rate K1_ATP in the myocardium in the adenosine stress state based on equation (3).

$${K}_{1 \_ ATP} = \frac{{L}_{a}}{{L}_{m}} \frac{{I}_{m} ( t ) ATP}{{\int }_{0}^{t} {I}_{a} ( τ ) d τ} \qquad ( 3 )$$

Subsequently, the processing circuitry 34 calculates a second blood flow ratio (K1_ATP/K1) in the myocardium by dividing the calculated second blood flow rate K1_ATP by the calculated zeroth blood flow rate K1.

### (Step ST40)

The processing circuitry 34 generates a first ratio image r1 having a pixel value corresponding to the first blood flow ratio (K1_ACh/K1) calculated for each pixel and a second ratio image r2 having a pixel value corresponding to the second blood flow ratio (K1_ATP/K1) calculated for each pixel. In addition, the processing circuitry 34 generates a zeroth blood flow rate image f0 having a pixel value corresponding to the zeroth blood flow rate K1 calculated for each pixel. Likewise, the processing circuitry 34 generates a first blood flow rate image f1 having a pixel value corresponding to the first blood flow rate K1_ACh calculated for each pixel and a second blood flow rate image f2 having a pixel value corresponding to the second blood flow rate K2_ATP calculated for each pixel.

### (Step ST50)

As indicated by the second row of FIG. 4, the processing circuitry 34 causes the display 32 to display the zeroth blood flow rate image f0, the first blood flow rate image f1, and the second blood flow rate image f2 side by side. In addition, as indicated by the third row of FIG. 4, the processing circuitry 34 causes the display 32 to display the first ratio image r1 and the second ratio image r2 side by side. The first ratio image r1 and the second ratio image r2 are an example of displaying the first blood flow ratio and the second blood flow ratio in the image form. The first ratio image r1 includes a vascular spasm region A1 having the first blood flow ratio which is relatively low in the image. The second ratio image r2 includes a vascular dilation region A2 having the second blood flow ratio which is relatively high in the image. The zeroth blood flow rate image f0, the first blood flow rate image f1, the second blood flow rate image f2, the first ratio image r1, and the second ratio image r2 are displayed in color by assigning a color corresponding to the pixel value of each pixel.

As indicated by the lower row of FIG. 4, the processing circuitry 34 causes the display 32 to display the zeroth blood flow rate K1 (for example, 3.0), the first blood flow rate K1_ACh (for example, 2.4), and the second blood flow rate K1_ATP (for example, 3.9) side by side as values in the myocardial ROI 202. Likewise, the processing circuitry 34 causes the display 32 to display the zeroth blood flow ratio (1.0), the first blood flow ratio (for example, 0.8 (=2.4/3.0)), and the second blood flow ratio (for example, 1.3 (=3.9/3.0)) side by side as values in the myocardial ROI 202.

Note that in the acetylcholine stress state, “no change” indicates normality, and “a change” (blood flow decrease) indicates abnormality. In the adenosine stress state, “no change” indicates abnormality, and “a change” (blood flow increase) indicates normality.

To elaborate further, in the acetylcholine stress state, no change in the first blood flow rate K1_ACh to the zeroth blood flow rate K1 indicates normality, and a decrease in the first blood flow rate K1_ACh indicates a disease. More specifically, in the acetylcholine stress state, if the first blood flow ratio exceeds the first threshold (for example, 0.5), the subject is normal, whereas if the first blood flow ratio is equal to or less than the first threshold, the subject has a disease. The disease detected by an acetylcholine stress examination corresponds to microvascular spasm among causes of INOCA.

In an adenosine stress examination, if the second blood flow rate K1_ATP does not change from the zeroth blood flow rate K1, the subject has a disease, whereas if the second blood flow rate K1_ATP increases, the subject is normal. More specifically, in an adenosine stress examination, if the second blood flow ratio is equal to or less than the second threshold (for example, 1.8), the subject has a disease, whereas if the second blood flow ratio exceeds the second threshold, the subject is normal. The disease detected in an adenosine stress examination corresponds to no microvascular dilation among the causes of INOCA.

Referring to FIG. 4, the hatching around the second blood flow ratio “1.3” is an example of alert display indicating abnormality if the second blood flow ratio in the myocardial ROI 202 is equal to or less than the second threshold (1.8).

Accordingly, as FIG. 4 shows an example, the operator can check blood flow rates and blood flow ratios together with the contrast-enhanced images g0 to g2, the blood flow rate images f0 to f2, and the ratio images r1 and r2 in the respective states, namely, the reference state of the subject, the acetylcholine stress state, and the adenosine stress state.

As described above, according to the first embodiment, the processing circuitry 34 acquires the zeroth contrast-enhanced image g0 when the heart of the subject is in the reference state, the first contrast-enhanced image g1 when the heart is in the acetylcholine stress state, and the second contrast-enhanced image g2 when the heart is in the adenosine stress state. The processing circuitry 34 calculates the first blood flow ratio representing the ratio between the zeroth blood flow rate K1 in the myocardium in the reference state and the first blood flow rate K1_ACh in the myocardium in the acetylcholine stress state based on the zeroth contrast-enhanced image g0 and the first contrast-enhanced image g1. The processing circuitry 34 calculates the second blood flow ratio representing the ratio between the zeroth blood flow rate K1 and the second blood flow rate K1_ATP in the myocardium in the adenosine stress state based on the zeroth contrast-enhanced image g0 and the second contrast-enhanced image g2. The processing circuitry 34 causes the display 32 to display the first blood flow ratio and the second blood flow ratio side by side. The arrangement configured to calculate blood flow ratios from contrast-enhanced images in this manner can quantitatively measure blood flows from contrast-enhanced images without using any sensor wire and display measurement results such as blood flow rates and blood flow ratios when performing an INOCA examination.

According to the first embodiment, the processing circuitry 34 causes the display 32 to display the first blood flow ratio and the second blood flow ratio at least in the numerical value form or the image form. In addition to the above effects, it is possible to display blood flow ratios in a desired form.

According to the first embodiment, the processing circuitry 34 sets the artery ROI 201 and the myocardial ROI 202 as regions of interest common to the zeroth contrast-enhanced image g0, the first contrast-enhanced image g1, and the second contrast-enhanced image g2. The processing circuitry 34 calculates the first blood flow ratio in each of the myocardial ROIs 202 in the zeroth contrast-enhanced image g0 and the first contrast-enhanced image g1. The processing circuitry 34 calculates the second blood flow ratio in each of the myocardial ROIs 202 in the zeroth contrast-enhanced image g0 and the second contrast-enhanced image g2. In the case of the numerical value form, the processing circuitry 34 causes the display to display the first blood flow ratio in the first display mode if the first blood flow ratio in the myocardial ROI 202 is equal to or less than the first threshold. Likewise, the processing circuitry 34 displays the second blood flow ratio in the second display mode if the second blood flow ratio in the myocardial ROI 202 is equal to or less than the second threshold. Accordingly, in addition to the above effects, it is possible to attract attention from the operator by display in the first display mode and the second display mode if the first blood flow ratio and the second blood flow ratio each are equal to or less than the threshold.

According to the first embodiment, the first display mode is a mode of performing color display or alert display to indicate an abnormality in the first blood flow ratio. The second display mode is a mode of performing color display or alert display to indicate an abnormality in the second blood flow ratio. Accordingly, in addition to the above effects, color display or alert display can attract more attention from the operator.

In addition, according to the first embodiment, the processing circuitry 34 calculates the first blood flow ratio for each corresponding pixel in the zeroth contrast-enhanced image g0 and the first contrast-enhanced image g1. The processing circuitry 34 calculates the second blood flow ratio for each corresponding pixel in the zeroth contrast-enhanced image g0 and the second contrast-enhanced image g2. The processing circuitry 34 generates the first ratio image r1 having a pixel value corresponding to the first blood flow ratio calculated for each pixel and the second ratio image r2 having a pixel value corresponding to the second blood flow ratio calculated for each pixel. In the case of the image form, the processing circuitry 34 causes the display 32 to display the first ratio image and the second ratio image in color by assigning colors to pixel values. Accordingly, in addition to the above effects, the arrangement configured to display a blood flow ratio in color for each pixel can easily visually present the operator with the distribution of blood flow ratios in ischemia with no-obstructive coronary artery disease.

According to the first embodiment, the processing circuitry 34 causes the display 32 to display at least the first blood flow rate K1_ACh and the second blood flow rate K1_ATP of the zeroth blood flow rate K1, the first blood flow rate K1_ACh, and the second blood flow rate K1_ATP side by side. In this case, in addition to the above effects, the operator can visually check the first blood flow rate K1_ACh and the second blood flow rate K1_ATP in the myocardium.

In addition, according to the first embodiment, the zeroth contrast-enhanced image g0, the first contrast-enhanced image g1, and the second contrast-enhanced image g2 are medical images obtained by radiography under almost the same radiography conditions except for the state of the heart. Accordingly, in addition to the above effects, in measuring blood flow ratios from a plurality of contrast-enhanced images, it is possible to suppress the influence of a change in the radiography conditions among the respective contrast-enhanced images.

### Modification of First Embodiment

In the first embodiment, the first blood flow ratio is calculated by calculating the zeroth blood flow rate K1 and the first blood flow rate K1_ACh. However, this is not exhaustive. For example, the processing circuitry 34 may obtain the first blood flow ratio (K1_ACh/K1) in the myocardium as the luminance ratio (Im(t)ACh/Im(t)) between the first contrast-enhanced image and the zeroth contrast-enhanced image as indicated by equation (4) without calculating the zeroth blood flow rate K1 and the first blood flow rate K1_ACh. More specifically, the processing circuitry 34 may calculate the first blood flow ratio by dividing the pixel values of corresponding pixels in the myocardial region in the zeroth contrast-enhanced image g0 and the myocardial region in the first contrast-enhanced image g1.

$$\frac{{K}_{1 \_ ACh}}{{K}_{1}} = \frac{\frac{{L}_{a}}{{L}_{m}} \frac{{I}_{m} ( t ) ACh}{{\int }_{0}^{t} {I}_{a} ( τ ) d τ}}{\frac{{L}_{a}}{{L}_{m}} \frac{{I}_{m} ( t )}{{\int }_{0}^{t} {I}_{a} ( τ ) d τ}} = \frac{{I}_{m} ( t ) ACh}{{I}_{m} ( t )} \qquad ( 4 )$$

In this case, in addition to the effects of the first embodiment, it is possible to reduce the stress and time taken to calculate the zeroth blood flow rate K1 and the first blood flow rate K1_ACh.

Likewise, in the first embodiment, the second blood flow ratio is calculated by calculating the zeroth blood flow rate K1 and the second blood flow rate K1_ATP. However, this is not exhaustive. For example, the processing circuitry 34 may obtain the second blood flow ratio (K1_ATO/K1) in the myocardium as the luminance ratio (Im(t)ATP/Im(t)) between the second contrast-enhanced image and the zeroth contrast-enhanced image as indicated by equation (5) without calculating the zeroth blood flow rate K1 and the second blood flow rate K1_ATP. More specifically, the processing circuitry 34 may calculate the second blood flow ratio by dividing the pixel values of corresponding pixels in the myocardial region in the zeroth contrast-enhanced image g0 and the myocardial region in the second contrast-enhanced image g2.

$$\frac{{K}_{1 \_ ATP}}{{K}_{1}} = \frac{\frac{{L}_{a}}{{L}_{m}} \frac{{I}_{m} ( t ) ATP}{{\int }_{0}^{t} {I}_{a} ( τ ) d τ}}{\frac{{L}_{a}}{{L}_{m}} \frac{{I}_{m} ( t )}{{\int }_{0}^{t} {I}_{a} ( τ ) d τ}} = \frac{{I}_{m} ( t ) ATP}{{I}_{m} ( t )} \qquad ( 5 )$$

In this case, likewise, in addition to the effects of the first embodiment, it is possible to reduce the stress and time taken to calculate the zeroth blood flow rate K1 and the second blood flow rate K1_ATP.

Likewise, the first embodiment is configured to calculate the first blood flow ratio and the second blood flow ratio with respect to the zeroth blood flow rate in the reference state. However, this is not exhaustive. For example, the calculation function 34c of the processing circuitry 34 may calculate the third blood flow ratio (K1_ATP/K1_ACh) by dividing the pixel values of corresponding pixels in the myocardial region in the first contrast-enhanced image g1 and the myocardial region in the second contrast-enhanced image g2. For example, the processing circuitry 34 may obtain the third blood flow ratio as the luminance ratio (Im(t)ATP/Im(t)ACh) between the second contrast-enhanced image g2 and the first contrast-enhanced image g1 as indicated by equation (6). Alternatively, the processing circuitry 34 may obtain the third blood flow ratio as the ratio between the second blood flow rate K1_ATP represented by equation (3) and the first blood flow rate K1_ACh represented by equation (2) as indicated by equation (6). When the third blood flow ratio is to be obtained, the first blood flow ratio and the second blood flow ratio may not be obtained or may be obtained as in the above case. The calculation function 34c is an example of the third calculation unit. The processing circuitry 34 causes the display 32 to further display the third blood flow ratio.

$$\frac{{K}_{1 \_ ATP}}{{K}_{1 \_ ACh}} = \frac{\frac{{L}_{a}}{{L}_{m}} \frac{{I}_{m} ( t ) ATP}{{\int }_{0}^{t} {I}_{a} ( τ ) d τ}}{\frac{{L}_{a}}{{L}_{m}} \frac{{I}_{m} ( t ) ACh}{{\int }_{0}^{t} {I}_{a} ( τ ) d τ}} = \frac{{I}_{m} ( t ) ATP}{{I}_{m} ( t ) ACh} \qquad ( 6 )$$

According to this modification, in addition to the effects of the first embodiment, blood flow ratios can be measured without using the data of the zeroth contrast-enhanced image g0 in the reference state. Note that reducing the data to be used eliminates the necessity of positioning and various types of linearization. This can implement faster and more stable processing.

The first embodiment is configured to display both the pair of first blood flow rate image f1 and second blood flow rate image f2 and the pair of first blood flow rate image f1 and second ratio image r2. However, this is not exhaustive. For example, the processing circuitry 34 may omit displaying the first ratio image r1 and the second ratio image r2. Alternatively, the processing circuitry 34 may omit displaying the first blood flow rate image f1 and the second blood flow rate image f2. In addition, the processing circuitry 34 may select as needed whether to display each of contrast-enhanced images, blood flow rate images, and ratio images in accordance with an operation by the operator. In the modification described above, it is possible to obtain the same effects as those of the first embodiment while simplifying display. In addition, this modification can be applied to the mode of displaying a blood flow rate instead of a blood flow ratio during display of only the blood flow ratio and hence can also be applied to another embodiment associated with the display range of ratio images and the graph display of blood flow ratios.

### Second Embodiment

The second embodiment is an embodiment provided with the display ranges of a first ratio image r1 and a second ratio image r2 unlike the first embodiment that is not provided with the display ranges of the first ratio image r1 and the second ratio image r2.

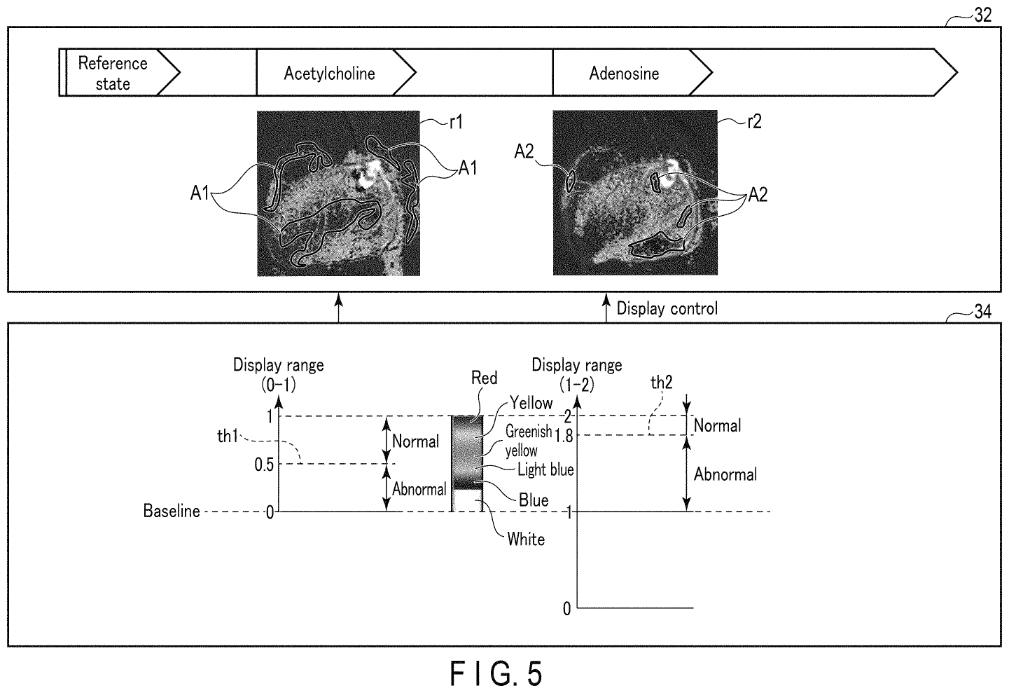

Accordingly, in addition to the above functions, a display control function 34e of a processing circuitry 34 has a function of assigning colors to pixel values so as to align the baseline (0) of the display range (0-1) of the first ratio image r1 with the baseline (1) of the display range (1-2) of the second ratio image r2, as FIG. 5 shows an example. In this case, the display range (0-1) of the first ratio image r1 corresponds to the first blood flow ratio range (0-1) in the acetylcholine stress state that causes microvascular spasm. This is because the first blood flow ratio becomes a value equal to or less than 1 along with microvascular spasm. The first ratio image r1 has 256 colors assigned to 256 pixel values corresponding to the first blood flow ratios of 0 to 1. The colors used are those obtained by changing from white to blue, light blue, greenish yellow, yellow, and red in 256 levels. Likewise, the display range (1-2) of the second ratio image r2 corresponds to the second blood flow ratio range (1-2) in the adenosine stress state that causes microvascular dilation. This is because the second blood flow ratio becomes a value equal to or more than 1 along with microvascular dilation. Note that the upper limit value of the second blood flow ratios sometimes exceeds 2. In this case, however, setting the upper limit value of the second blood flow ratios to 2 will normalize the second blood flow ratios within the range of 1 (inclusive) to 2 (inclusive). The second ratio image r2 has 256 colors assigned to 256 pixel values corresponding to the second blood flow ratios normalized within the range of 1 (inclusive) to 2 (inclusive).

Other arrangements are similar to those in the first embodiment.

According to the above arrangement, the processing circuitry 34 executes steps ST10 to ST50 in the same manner as described above. In step ST50, however, the processing circuitry 34 causes a display 32 to display the first ratio image r1 and the second ratio image r2 upon aligning the baselines of the display ranges. This allows the operator to visually check the distribution of the first blood flow ratios in the first ratio image r1 and the distribution of the second blood flow ratios in the second ratio image r2 by color display in the same display range.

As described above, according to the second embodiment, the processing circuitry 34 assigns colors to pixel values so as to align the baseline of the display range of the first ratio image r1 with the baseline of the display range of the second ratio image r2. In addition to the above effects, this makes it possible to display the first ratio image r1 and the second ratio image r2 in colors while the baselines of the display ranges are aligned with each other.

### Modification of Second Embodiment

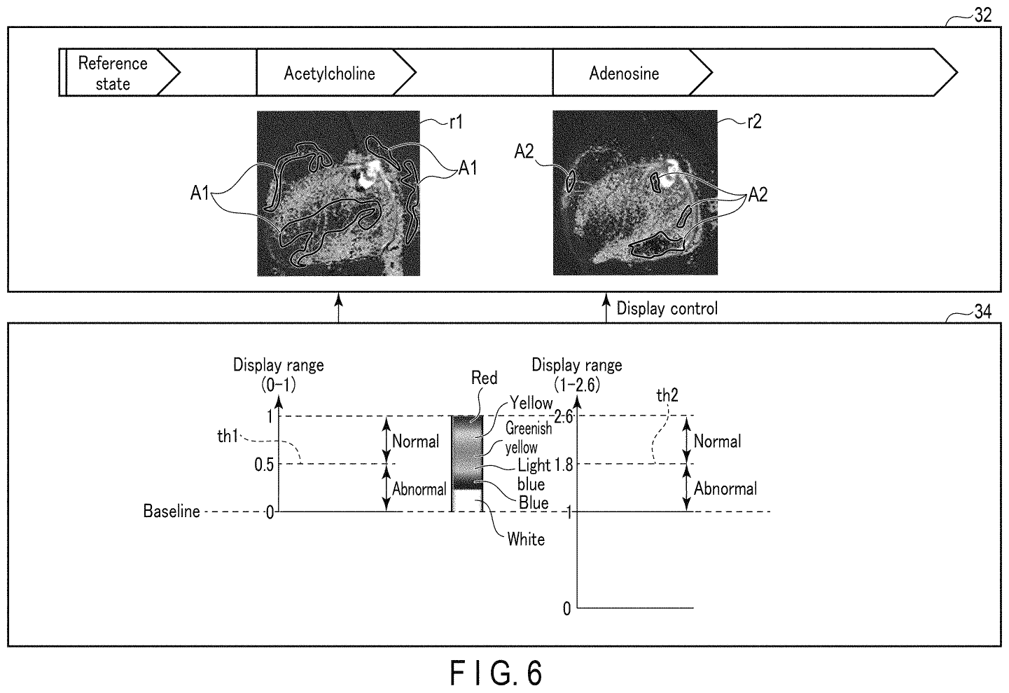

In the second embodiment, the baselines of the display ranges of the first ratio image r1 and the second ratio image r2 are aligned with each other. However, this is not exhaustive. For example, as shown in FIG. 6, the processing circuitry 34 may cause the display 32 to display the first ratio image r1 and the second ratio image r2 so as to assign the same color to a pixel value corresponding to a first threshold th1 for discriminating the first blood flow ratios in the first ratio image r1 into normality and abnormality and a pixel value corresponding to a second threshold th2 for discriminating the second blood flow ratios in the second ratio image r2 into normality and abnormality.

In this case, as described above, the first ratio image r1 has the display range (0-1). In contrast to this, the display range (1-2.6) of the second ratio image r2 is set by normalizing the second blood flow ratios within the range of 1 (inclusive) to 2.6 (inclusive) by setting the upper limit value of the second blood flow ratios to 2.6 so as to match the second threshold th2 with the first threshold th1. The second ratio image r2 according to the modification has 256 colors to 256 pixel values corresponding to the second blood flow ratios normalized within the range of 1 (inclusive) to 2.6 (inclusive).

Other arrangements are similar to those in the first embodiment.

According to the above modification of the second embodiment, the processing circuitry 34 executes steps ST10 to ST50 in the same manner as described above. In step ST50, however, the processing circuitry 34 causes the display 32 to display the first ratio image r1 and the second ratio image r2 so as to align the baselines of the display ranges and match the first threshold with the second threshold. This allows the operator to visually check the abnormal region of the first blood flow ratios in the first ratio image r1 and the abnormal region of the second blood flow ratios in the second ratio image r2 by color display of the same color. Accordingly, in addition to the effects of the second embodiment, it is possible to more easily visually check whether each blood flow ratio in the first ratio image r1 and the second ratio image r2 is abnormal.

### Third Embodiment

The third embodiment is an embodiment configured to display a first graph representing the first blood flow ratio and a second graph representing the second blood flow ratio unlike the second embodiment associated with the display ranges of the first ratio image r1 and the second ratio image r2.

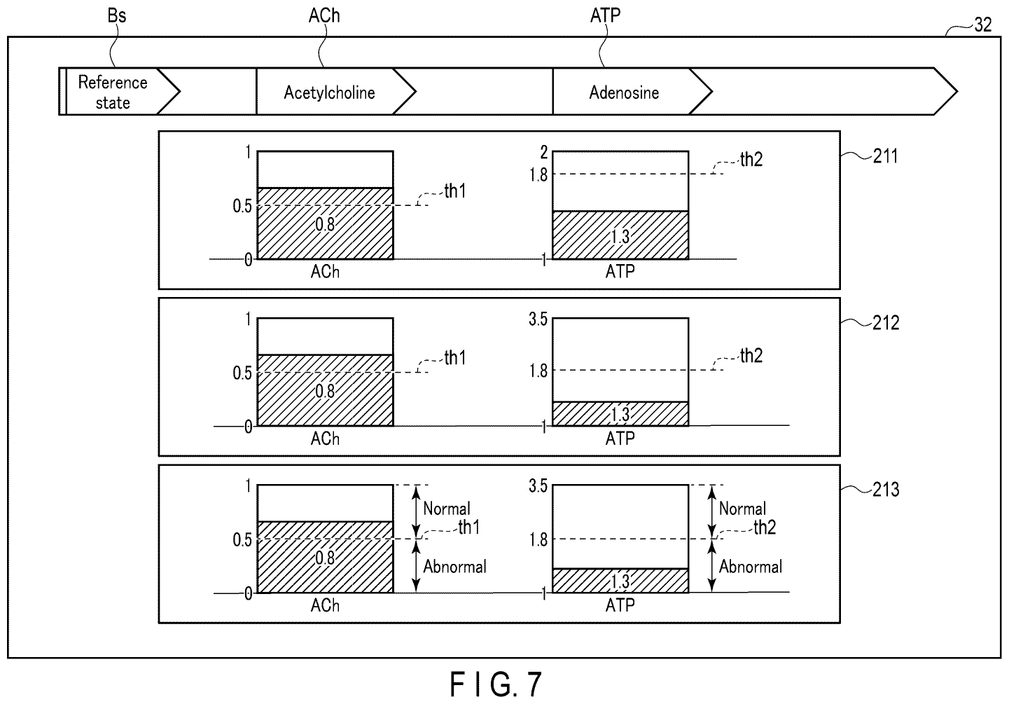

Accordingly, as shown in FIG. 7, in addition to the above functions, a display control function 34e of a processing circuitry 34 causes a display 32 to display the first graph representing the first blood flow ratio and a first threshold th1 in a myocardial ROI 202 and the second graph representing the second blood flow ratio and a second threshold th2 in the myocardial ROI 202 side by side. For example, the processing circuitry 34 may cause the display 32 to display the first graph and the second graph in any one of the modes in display regions 211 to 213.

The display region 211 is configured to display the first graph and the second graph side by side so as to align the baseline of the description range (0-1) of the first graph with the baseline of the description range (1-2) of the second graph. The display region 211 corresponds to the mode shown in FIG. 5.

The display region 212 is configured to display the first graph and the second graph side by side so as to align the first threshold th1 in the first graph with the second threshold th2 in the second graph. That is, the description range (0-1) of the first graph is similar to the display region 211. The description range (1-3.5) of the second graph is set by normalizing the second blood flow ratios within the range of 1 (inclusive) to 3.5 (inclusive) by setting the upper limit value of the second blood flow ratios to 3.5 so as to match the second threshold th2 with the first threshold th1. The display region 212 corresponds to the mode shown in FIG. 6 described above.

The display region 213 is configured to display a normal range and an abnormal range in the first graph, with the first threshold th1 serving as a boundary and to display a normal range and an abnormal range in the second graph, with the second threshold th2 serving as a boundary.

Other arrangements are similar to those of the second embodiment.

With the above arrangement, the processing circuitry 34 executes steps ST10 to ST50 in the same manner as described above. In step ST50, however, the processing circuitry 34 causes the display 32 to display the first graph representing the first blood flow ratio and the first threshold th1 in the myocardial ROI 202 and the second graph representing the second blood flow ratio and the second threshold th2 in the myocardial ROI 202 side by side. For example, the processing circuitry 34 causes the display 32 to display the first graph and the second graph in any one of the modes indicated by the display regions 211 to 213. This allows the operator to visually check the first graph representing the first blood flow ratio and the first threshold th1 and the second graph representing the second blood flow ratio and the second threshold th2.

As described above, according to the third embodiment, the processing circuitry 34 causes the display to display the first graph representing the first blood flow ratio and the first threshold in the myocardial ROI 202 and the second graph representing the second blood flow ratios and the second threshold in the myocardial ROI 202 side by side. Accordingly, in addition to the above effects, the operator can visually check the respective blood flow ratios and the thresholds th1 and th2 in the bar graph mode. Referring to FIG. 7, the first graph and the second graph are bar graphs. However, this is not exhaustive. Note, however, that the first graph and the second graph are preferably the same type of graph from the viewpoint of facilitating comparison.

In addition, according to the third embodiment, the processing circuitry 34 may cause the display to display the first graph and the second graph side by side so as to align the baseline of the description range of the first graph with the baseline of the description range of the second graph. In this case, it is possible to display the first graph and the second graph side by side while the baselines of the description ranges are aligned with each other.

According to the third embodiment, the processing circuitry 34 may cause the display to display the first graph and the second graph side by side so as to align the first threshold in the first graph with the second threshold in the second graph. In this case, it is possible to more easily visually check whether each blood flow ratio in the first graph and the second graph is abnormal.

In addition, according to the third embodiment, the processing circuitry 34 may cause the display to display a normal range and an abnormal range in the first graph, with the first threshold th1 serving as a boundary, and to display a normal range and an abnormal range in the second graph, with the second threshold th2 serving as a boundary. In this case, it is possible to more easily visually check whether each blood flow ratio in the first graph and the second graph is abnormal.

### Fourth Embodiment

The fourth embodiment is an embodiment configured to display the first blood flow ratio and the second blood flow ratio in one graph unlike the third embodiment configured to display a plurality of graphs side by side.

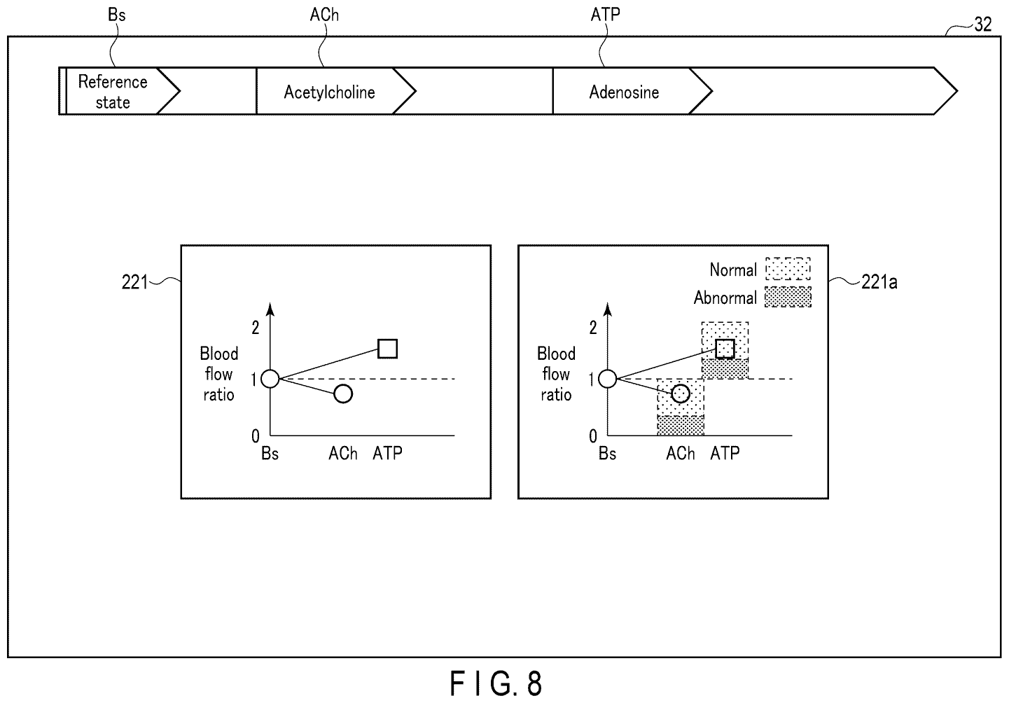

Accordingly, in addition to the above functions, a display control function 34e of a processing circuitry 34 causes a display 32 to display a comparison graph that comparatively shows each of the first and second blood flow ratios in a myocardial ROI 202 relative to a reference value when the blood flow ratio (K1/K1) at a zeroth blood flow rate K1 is 1 as the reference value. For example, the processing circuitry 34 may cause the display 32 to display the comparison graph in any of the modes indicated by comparison graphs 221 and 221a, as shown in FIG. 8.

In this case, the comparison graph 221 represents a blood flow ratio for each state, with the ordinate representing the blood flow ratio and the abscissa representing the state. More specifically, in the comparison graph 221, the first blood flow ratio (the white circle in FIG. 8) in an acetylcholine stress state ACh and the second blood flow ratio (the white rectangle in FIG. 8) in an adenosine stress state ATP are connected to the reference value (1) in a reference state Bs with straight lines.

The comparison graph 221a is a mode of displaying a normal range and an abnormal range with respect to the first blood flow ratio and the second blood flow ratio in the comparison graph 221.

Other arrangements are similar to those in the third embodiment.

According to the above arrangement, the processing circuitry 34 executes steps ST10 to ST50 in the same manner as described above. In step ST50, however, the processing circuitry 34 causes the display 32 to display the comparison graphs 221 and 221a comparatively showing each of the first and second blood flow ratios in the myocardial ROI 202 with respect to a reference value when the blood flow ratio (K1/K1) at the zeroth blood flow rate K1 is 1 as the reference value. Accordingly, the operator can visually check the comparison graphs 221 and 221a representing each of the first and second blood flow ratios with respect to the reference value.

As described above, according to the fourth embodiment, the processing circuitry 34 causes the display 32 to display the comparison graphs 221 and 221a comparatively showing each of the first and second blood flow ratios in the myocardial ROI 202 with respect to a reference value when the blood flow (K1/K1) at the zeroth blood flow rate K1 is 1 as the reference value. This makes it possible to comparatively visually check two blood flow ratios in one comparison graph 221 or 221a.

In addition, according to the fourth embodiment, the processing circuitry 34 can display the comparison graph 221a in the mode of displaying a normal range and an abnormal range with respect to each of the first and second blood flow ratios in the comparison graph 221. In addition to the above effects, this makes it possible to visually check whether two blood flow ratios are in the normal rage or the abnormal range in the comparison graph 221a.

### Modification of Fourth Embodiment

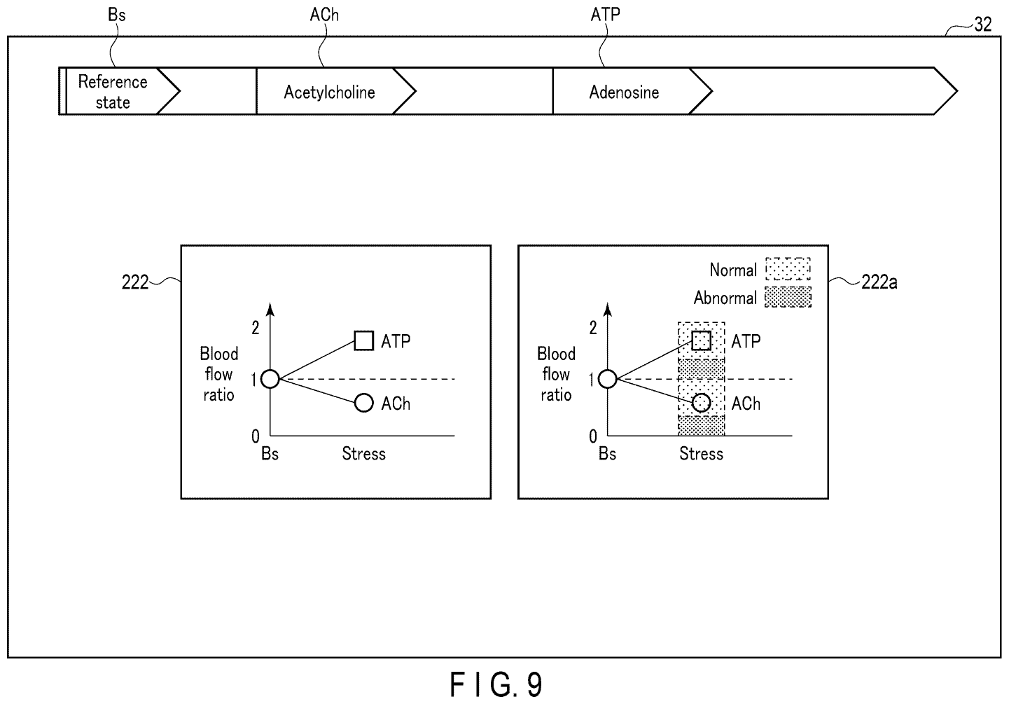

In the fourth embodiment, the abscissa of each of the comparison graphs 221 and 221a represents the state. However, this is not exhaustive. For example, as shown in FIG. 9, the processing circuitry 34 may cause the display 32 to display a comparison graph 222 or 222a in a mode with the abscissa of the comparison graph 221 or 221a representing the stress. Since the abscissa of each of the comparison graphs 222 and 222a represents the stress, “ACh” representing an acetylcholine stress state is placed near the white circle representing the first blood flow ratio, and “ATP” representing an adenosine stress state is placed near the white rectangle representing the second blood flow ratio. Such modification can also obtain effects similar to those of the fourth embodiment.

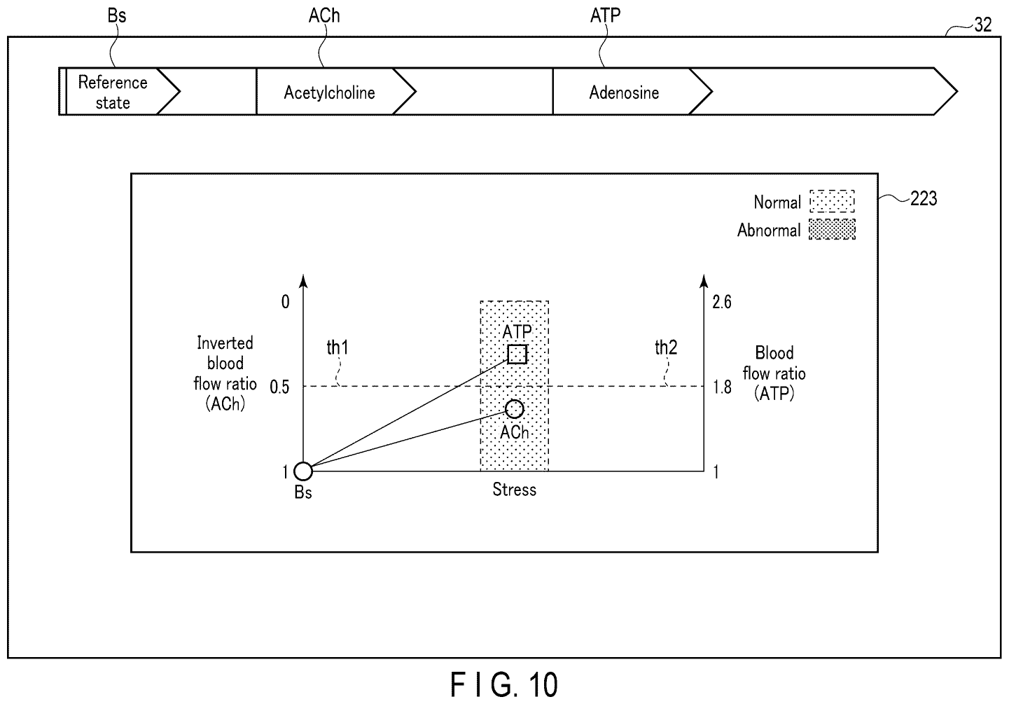

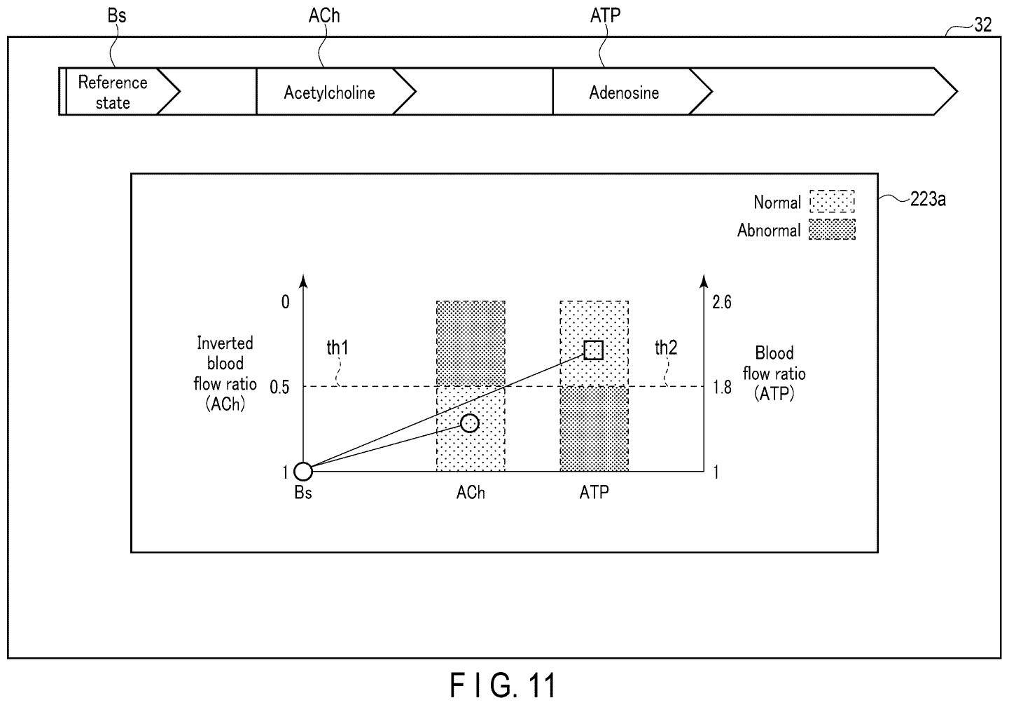

In the modification of the fourth embodiment, in each of the comparison graphs 222 and 222a, the first and second blood flow ratios are separated vertically from the reference value “1”. However, this is not exhaustive. For example, as shown in FIGS. 10 and 11, the processing circuitry 34 may cause the display to display the first blood flow ratios in comparison graphs 223 and 223a while the description range is reversed centered on the reference value and display thresholds th1 and th2 with respect to each of the first and second blood flow ratios in the comparison graphs 223 and 223a. The processing circuitry 34 may cause the display to display a normal range and an abnormal range with respect to each of the first and second blood flow ratios. Such modification makes it possible to visually check the comparison graph 223 in a mode with each of the first and second blood flow ratios extending upward from the reference value “1”.

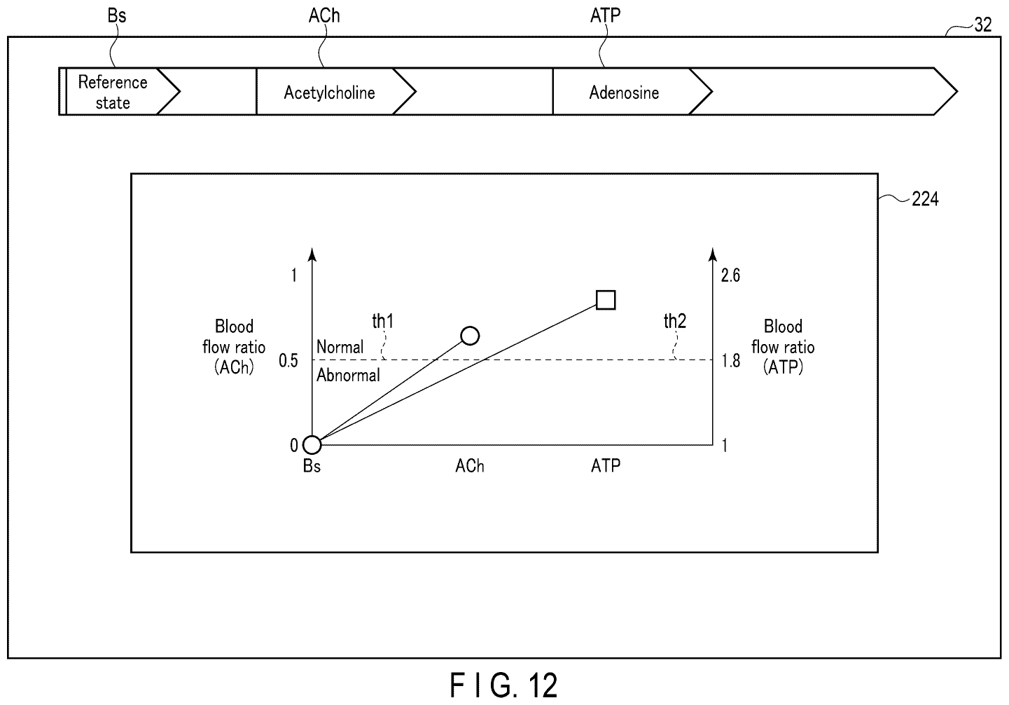

The modification of the fourth embodiment is configured to display a normal range and an abnormal range with respect to each of the first and second blood flow ratios in the comparison graph 223a. However, this is not exhaustive. For example, as shown in FIG. 12, the processing circuitry 34 may cause the display to display the first blood flow ratio in a comparison graph 224, with the reference value “1” being placed at the lower end and the reverse of the description range being returned (with the state of the lower side being 0 and that of the upper side being 1), and also display the thresholds th1 and th2 for each of the first and second blood flow ratios in the comparison graph 224. Such modification makes it possible to visually check the comparison graph 224 that is a mode having a normal range and an abnormal range common to the first and second blood flow rations, with each of the first and second blood flow ratios extending upward from the reference value “1”.

### Fifth Embodiment

The fifth embodiment is an embodiment configured to determine whether the cause of INOCA is the blood vessel or the myocardium unlike the first to fourth embodiments associated with the display of blood flow ratios. More specifically, the fifth embodiment is configured to perform determination upon discriminating causes of INOCA into coronary spasm of epicardial coronary artery and coronary microvascular spasm (MVS: microvascular spasm).

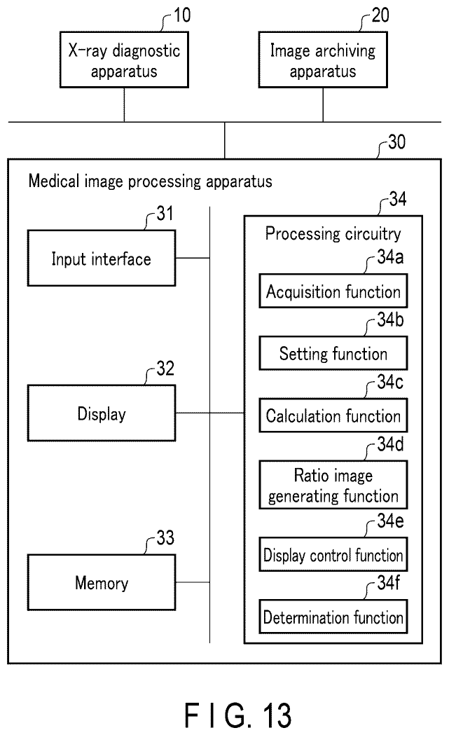

In this case, as shown in FIG. 13, a processing circuitry 34 further includes a determination function 34f. The determination function 34f determines, based on a myocardial blood flow image, whether the myocardial blood flow in the subject has decreased and also determines, based on the first contrast-enhanced image, whether the blood vessel in the subject is coarctated. Upon determining that the myocardial blood flow has decreased and the blood vessel is coarctated, the processing circuitry 34 detects coronary spasm. Upon determining that the myocardial blood flow has decreased and the blood vessel is not coarctated, the processing circuitry 34 determines microvascular spasm. The determination function 34f is an example of a determination unit.

Accordingly, in addition to the above functions, an acquisition function 34a of the processing circuitry 34 further acquires a myocardial blood flow image when the heart of the subject is in the acetylcholine stress state. A myocardial blood flow image is, for example, an electrocardiogram gated coronary artery contrast-enhanced image obtained by X-ray angiography using an X-ray diagnostic apparatus 10. The acquisition destination is, for example, the image archiving apparatus 20.

In addition to the above function, a display control function 34e of the processing circuitry 34 causes a display 32 to display a detection result.

Other arrangements are similar to those in the first embodiment.

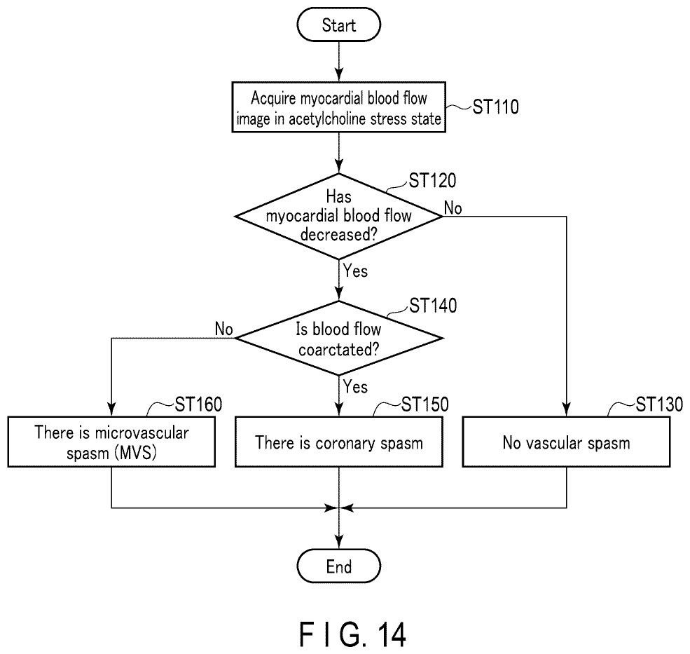

The operation of the medical image processing apparatus having the above arrangement will be described next with reference to the flowchart of FIG. 14. Assume that an image archiving apparatus 20 has saved the myocardial blood flow images acquired in advance by the X-ray diagnostic apparatus 10. A myocardial blood flow image is an electrocardiogram gated coronary artery contrast-enhanced image obtained by X-ray angiography when the heart of the subject is in the acetylcholine stress state.

As shown in FIG. 14, the processing circuitry 34 of a medical image processing apparatus 30 acquires a myocardial blood flow image when the heart of the subject is in the acetylcholine stress state from the image archiving apparatus 20 in accordance with an operation by the operator (step ST110) and saves the myocardial blood flow image in a memory 33.

The processing circuitry 34 determines, based on the myocardial blood flow image, whether the myocardial blood flow in the subject has decreased (step ST120). If NO, the processing circuitry 34 detects that there is no vascular spasm (step ST130) and causes a display 32 to display the detection result.

Upon determining in step ST120 that the myocardial blood flow has decreased, the processing circuitry 34 determines, based on the first contrast-enhanced image g1 described above, whether the blood vessel in the subject is coarctated (step ST140).

Upon determining in step ST140 that the blood vessel is coarctated, the processing circuitry 34 detects that the subject has coronary spasm (step ST150) and causes the display 32 to display the detection result. The processing is then terminated.

Upon determining in step ST140 that the blood vessel is not coarctated, the processing circuitry 34 detects that microvascular spasm (MVS) has occurred in the heart of the subject (step ST160) and causes the display 32 to display the detection result. The processing is then terminated.

Accordingly, the operator can visually check a display screen for the detection result and grasp the presence/absence of vascular spasm, the presence/absence of coronary spasm, and the presence/absence of microvascular spasm concerning the heart of the subject.

As described above, according to the fifth embodiment, the processing circuitry 34 further acquires a myocardial blood flow image when the heart is in the acetylcholine stress state. The processing circuitry 34 determines, based on the myocardial blood flow image, whether the myocardial blood flow in the subject has decreased and also determines, based on the first contrast-enhanced image, whether the blood vessel in the subject is coarctated. Upon determining that the myocardial blood flow has decreased and the blood vessel is coarctated, the processing circuitry 34 detects coronary spasm. Upon determining that the myocardial blood flow has decreased and the blood vessel is not coarctated, the processing circuitry 34 detects microvascular spasm. The processing circuitry 34 causes the display 32 to display the detection result. Accordingly, in addition to the above effects, it is possible to detect coronary spasm or microvascular spasm in the heart of the subject based on the myocardial blood flow image and the first contrast-enhanced image when the heart is in the acetylcholine stress state.

### Sixth Embodiment

The sixth embodiment is an embodiment configured to display blood flow rates, transit times, and blood flow ratios unlike the first embodiment configured to display blood flow rates and blood flow ratios.

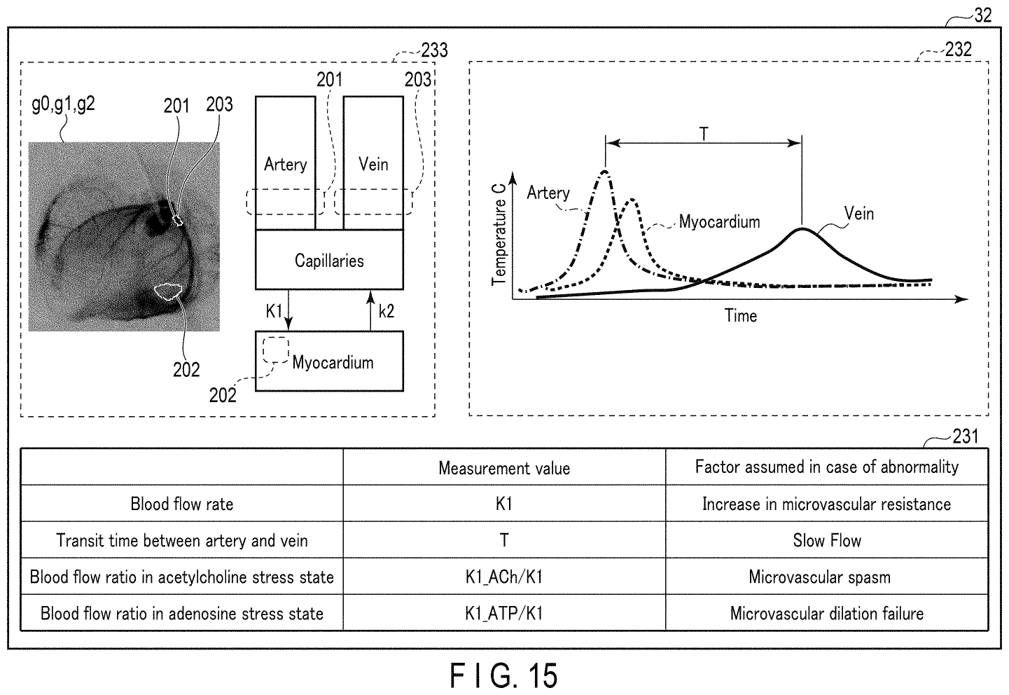

More specifically, for example, in addition to the above functions, as shown in FIG. 15, a processing circuitry 34 causes a display 32 to display a measurement value display region 231, a time density curve 232, and a schematic view 233.

The measurement value display region 231 is a region for displaying a measurement value of an index associated with the blood flow in the heart and an assumed factor, if the measurement value is abnormal, in association with each other. A measurement value includes a blood flow rate (K1) in the reference state, a transit time between the artery and the vein, the first blood flow ratio (K1_ACh/K1) in the acetylcholine stress state, and the second blood flow ratio (K1_ATP/K1) in the adenosine stress state.

Factors include, for example, a microvascular resistance increase associated with a blood flow rate (K1), Slow Flow associated with a transit time T, microvascular spasm associated with the first blood flow ratio (K1_ACh/K1), and a microvascular dilation failure associated with the second blood flow ratio (K1_ATP/K1). Not that Slow Flow is, for example, an index associated with the flow velocity of a contrast medium and can be expressed by the reciprocal of the transit time T (note that, distance=velocity×time).

Referring to the time density curve 232, the ordinate represents a contrast medium density C, and the abscissa represents the time. The time density curve 232 represents the time dependency of the contrast medium density C in an artery ROI 201 shown in the schematic view 233, the time dependency of the contrast medium density C in a myocardial ROI 202, and the time dependency of the contrast medium density C in a vein ROI 203. The time density curve 232 obtains the time dependency of the contrast medium density C in the vein ROI 203, and hence a longer time than usual is used for measurement. This makes it possible to obtain the transit time T of a contrast medium between the artery and the vein as the time difference between the time of the peak of the contrast medium density in the artery from the time density curve 232 and the time of the peak of the contrast medium density in the vein. The transit time T is displayed in the measurement value display region 231.

The schematic view 233 shows the artery ROI 201, the myocardial ROI 202, and the vein ROI 203 in contrast-enhanced images g0 to g2. In addition, the schematic view 233 schematically shows the artery, the capillaries, the myocardium, and the vein.

To elaborate further, the time density curve 232 is measured for each frame of a contrast-enhanced image, each pixel, or each region of interest. Equation (7) can be obtained from a two-compartment model.

$$\frac{{dC}_{m} ( t )}{dt} = {K}_{1} {C}_{a} ( t ) - {k}_{2} {C}_{m} ( t ) \qquad ( 7 )$$

In this case, referring to equation (7) and the schematic view 233, K1 represents the transfer constant of a contrast medium from the artery to the myocardial region, the unit is mL/min/g, k2 represents the transfer constant of the contrast medium from the myocardial region to the vein, Ca(t) represents a contrast medium density in the artery and corresponds to the curve indicated by “artery” in the time density curve 232, Cm(t) represents the contrast medium density in the myocardial region and corresponds to the curve indicated by “myocardium” in the time density curve 232, and K1 also represents the blood flow rate described above.

In an early period after the injection of a contrast medium, the inflow of the contrast medium from the artery to the myocardium is much larger than the outflow of the contrast medium from the myocardium to the vein, and hence K1Ca(t)>>k2Cm(t) can be assumed. Therefore, equation (7) can be converted into equation (8).

$${C}_{m} ( t ) = {K}_{1} {\int }_{0}^{t} {C}_{a} ( τ ) d τ \qquad ( 8 )$$

In this case, if the contrast medium densities Ca(t) and Cm(t) can be measured from the image, K1 can be calculated. In coronary artery X-ray angiography, since an X-ray image is a planar image, only integral information along the X-ray path can be measured, and a contrast medium density C(t) cannot be directly measured.

However, if it is assumed that the contrast medium density C(t) is an average density along an X-ray path L, I(t)=C(t)L can be measured from the X-ray image. In this case, I(t) represents an image luminance in the X-ray image having undergone baseline subtraction. In this case, the X-ray path L can be expressed as a path length La of the artery in unit of cm and a path length Im of the myocardium in unit of cm. Accordingly, the equation of image luminance I(t)=C(t) can be expressed as, for example, the equation of image luminance Im(t)=Cm(t)Lm of the myocardium and the equation of image luminance Ia(t)=Ca(t)La of the artery. In addition, equation (8) can be converted into equation (9) by using the relationship between contrast medium densities Cm(t)=Im(t)/Lm and Ca(t)=Ia(t)/La obtained by modifying the equations of the image luminances of the myocardium and the artery.

$${I}_{m} ( t ) = {K}_{1} \frac{{L}_{m}}{{L}_{a}} {\int }_{0}^{t} {I}_{a} ( τ ) d τ \qquad ( 9 )$$

Note that equation (1) described above can be obtained from equation (9).

Other arrangements are the same as those in the first embodiment.

According to the above arrangement, the processing circuitry 34 executes steps ST10 to ST50 in the same manner as described above. However, in step ST50, the processing circuitry 34 causes the display 32 to display the measurement value display region 231, the time density curve 232, and the schematic view 233. This allows the operator to visually check the relationships between four indices in the measurement value display region 231, the time density curve 232, and each ROI in the schematic view 233.

As described above, according to the sixth embodiment, the processing circuitry 34 causes the display 32 to display the blood flow rate (K1) in the reference state, the transit time T between the artery and the vein, the first blood flow ratio (K1_ACh/K1) in the acetylcholine stress state, and the second blood flow ratio (K1_ATP/K1) in the adenosine stress state. Accordingly, in addition to the above effects, the operator can visually check the transit time T between the artery and the vein.

In addition, according to the sixth embodiment, the arrangement configured to display the measurement value of an index associated with the blood flow in the heart and a factor assumed when the measurement value is abnormal in association with each other can support to assume a factor if the measurement value is abnormal.

### Seventh Embodiment

The seventh embodiment is an embodiment in which a medical image processing apparatus 30 mounted in an X-ray diagnostic apparatus 10 acquires contrast-enhanced images and calculates and displays blood flow ratios unlike the first embodiment in which the medical image processing apparatus 30 acquires images from the external image archiving apparatus 20.

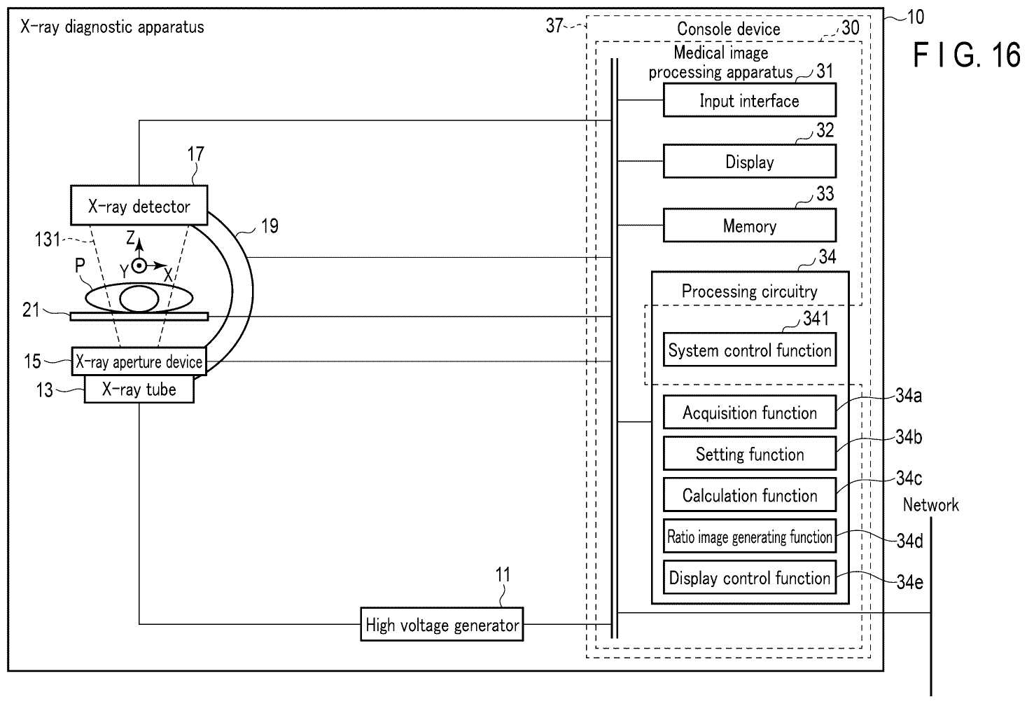

FIG. 16 shows an example of the arrangement of the X-ray diagnostic apparatus 10 according to the seventh embodiment. The X-ray diagnostic apparatus 10 includes a high voltage generator 11, an X-ray tube 13, an X-ray aperture device 15, an X-ray detector 17, a support frame 19, a bed (not shown) having a top 21, an input interface 31, a display 32, a memory 33, and a processing circuitry 34. The processing circuitry 34 includes a system control function 341, an acquisition function 34a, a setting function 34b, a calculation function 34c, a ratio image generating function 34d, and a display control function 34e. In this case, the input interface 31, the display 32, the memory 33, and the processing circuitry 34 constitute a console device 37. The arrangement obtained by omitting the system control function 341 from the console device 37 corresponds to the medical image processing apparatus 30.

In this case, the high voltage generator 11 generates a tube current supplied to the X-ray tube 13 and a tube voltage (high voltage) applied to the X-ray tube 13. The high voltage generator 11 supplies tube currents suitable for radiography and radioscopy to the X-ray tube 13 in accordance with radiography conditions under the control of the system control function 341 of the processing circuitry 34. The high voltage generator 11 supplies tube voltages suitable for radiography and radioscopy to the X-ray tube 13 in accordance with radiography conditions under the control of the system control function 341. When applying a tube voltage, the high voltage generator 11 may use a scheme of applying a tube voltage to the X-ray tube 13 temporally continuously or a scheme of applying a pulsed high voltage to the X-ray tube 13 by high voltage switching (to be referred to as a high voltage pulse application scheme). The following description is based on the assumption that the high voltage generator 11 performs radioscopy by using the high voltage pulse application scheme.

The X-ray tube 13 generates X-rays from an X-ray focus (to be referred to as a tube focus hereinafter) based on the tube current supplied from the high voltage generator 11 and the tube voltage applied by the high voltage generator 11. The X-rays generated from the tube focus are applied to a subject P while X-rays in an unnecessary region are blocked by the X-ray aperture device 15. A maximum irradiation range 131 of X-rays is indicated by the dotted lines. Assume that the X-ray tube 13 according to this embodiment is a rotating anode type X-ray tube. Note that the X-ray tube 13 according to the embodiment may be another type of X-ray tube such as a fixed anode type X-ray tube. The X-ray tube 13 generates pulse X-rays discreted at predetermined time intervals accompanying the application of pulsed high voltages by high voltage switching. The X-ray radiation window of the X-ray tube 13 is attached with lead cone that blocks extra-focal X-rays generated outside the tube focus.

The X-ray aperture device 15 is provided on the front surface of the X-ray tube 13 so as to be adjacent to the X-ray radiation window of the X-ray tube 13. The X-ray aperture device 15 limits the irradiation range of X-rays generated at the tube focus. Note that the X-ray aperture device 15 may have various types of filters (a radiation quality adjusting filter, an additional filter, a radiation quality reduction filter, and the like) in addition to the X-ray filter.

The X-ray detector 17 faces the X-ray tube 13 and detects the X-rays generated from the X-ray tube 13. The X-ray detector 17 is implemented by, for example, a flat panel detector (to be referred to as the FPD hereinafter). The FPD has a plurality of semiconductor detection elements. Note that an image intensifier may be used as the X-ray detector 17. The electrical signals generated by the plurality of semiconductor detection elements accompanying the application of X-rays are output to an analog to digital converter (not shown) (to be referred to as the A/D converter hereinafter). The A/D converter converts an electrical signal into digital data. The A/D converter outputs the digital data to the processing circuitry 34.

The support frame 19 movably supports the X-ray tube 13 and the X-ray detector 17. More specifically, the support frame 19 is a C-arm. The X-ray tube 13 and the X-ray detector 17 are mounted on the C-arm so as to face each other. A support pillar (not shown) supports the C-arm through a guide rail, a linear motion bearing, and the like so as to allow the C-arm to slide in a direction (to be referred to as the first direction hereinafter) along the C shape of the C-arm. The support pillar is provided on the floor surface of an examination room. The support pillar supports the C-arm through a bearing and the like so as to allow the C-arm to rotate in a direction (to be referred to as the second direction hereinafter) orthogonal to the first direction. Note that the support pillar can also support the C-arm through a bearing and the like so as to allow the C-arm to be translated in the short-axis direction (X-axis) and the long-axis direction (Y direction) of the top 21. In addition, the C-arm supports the X-ray tube 13 and the X-ray detector 17 through, for example, a guide rail and a linear motion bearing so as to be able to change the distance (the source image distance (to be referred to as the SID hereinafter)) between the tube focus of the X-ray tube 13 and the center of the X-ray detector 17.

Note that as the support frame 19, an Q-arm may be used instead of the C-arm or, for example, two arms (for example, robot arms) may be used to independently support the X-ray tube 13 and the X-ray detector 17. In addition, the support frame 19 may have a biplane structure including a C-arm and an Q-arm.

The bed (not shown) movably supports the top 21 (also called a decubitus bed) on which the subject P is placed. The subject P is placed on the top 21.

A drive device (not shown) drives, for example, the support frame 19 and the bed. The drive device includes, for example, a motor and a transmission mechanism (for example, a chain drive, a belt drive, a ball screw, or the like) that transmits the force generated by the motor to each type of unit to be driven. The drive device slides the support frame 19 in the first direction and rotates the support frame 19 in the second direction in accordance with drive signals corresponding to control signals output from the processing circuitry 34. Note that the drive device may rotate the X-ray detector 17 about the SID as a rotational axis under the control of the system control function 341.

The drive device moves the top 21 by driving the top 21 under the control of the system control function 341. With this operation, at the time of radiography or radioscopy, the subject P placed on the top 21 is placed between the X-ray tube 13 and the X-ray detector 17. More specifically, the drive device slides the top 21 in the short-axis direction (X-axis direction) of the top 21 and the long-axis direction (Y-axis direction) of the top 21 through the bearing, the guide rail, the linear motion bearing, and the like based on control signals output from the processing circuitry 34. In addition, the drive device lifts and lowers the top 21 in the vertical direction (Z-axis direction) through the bearing, the guide rail, the linear motion bearing, and the like. In addition, the drive device may rotate the top 21 through the bearing, the guide rail, the linear motion bearing, and the like in order to tilt the top 21 along at least one of the long-axis direction and the short-axis direction as a rotational axis.

The input interface 31 inputs radiography conditions, a fluoroscopy position, a radiography range (imaging field of view of) of X-rays, the position and size of a region of interest in an X-ray image, and the like in accordance with instructions from the operator.

The processing circuitry 34 reads out various types of programs for controlling various circuits in the X-ray diagnostic apparatus 10, the drive device, and the like from the memory 33 and executes the read programs to implement various functions. The processing circuitry 34 temporarily stores information such as instructions from the operator which are sent from the input interface 31, radiography conditions such as radiography conditions, and fluoroscopy conditions in a memory (not shown). The processing circuitry 34 controls the high voltage generator 11, the X-ray aperture device 15, the drive device, and the like to cause the system control function 341 to execute radiography and radioscopy (pulse radiography) in accordance with instructions from the operator, a fluoroscopy/radiography position, and radiography conditions stored in the memory.

The acquisition function 34a, the setting function 34b, the calculation function 34c, the ratio image generating function 34d, and the display control function 34e of the processing circuitry 34 function in the same manner as described above. The acquisition function 34a acquires the zeroth contrast-enhanced image g0, the first contrast-enhanced image g1, and the second contrast-enhanced image g2 from the memory 33 instead of the image archiving apparatus 20.

According the above arrangement, the X-ray diagnostic apparatus 10 executes radiography based on radiography conditions. That is, the X-ray tube 13 irradiates the subject P into which a contrast medium is injected with X-rays. The X-ray detector 17 detects the X-rays applied from the X-ray tube 13 and transmitted through the subject P and outputs the X-ray detection result. The processing circuitry 34 generates a contrast-enhanced image of the subject P and saves the contrast-enhanced image in the memory 33 based on the output from the X-ray detector 17.

As described above, the X-ray diagnostic apparatus 10 generates and saves the zeroth contrast-enhanced image g0 when the heart having INOCA is in the reference state, the first contrast-enhanced image g1 when the heart is in the acetylcholine stress state, and the second contrast-enhanced image g2 when the heart is in the adenosine stress state.

Subsequently, steps ST10 to ST50 are executed in the same manner as described above. The processing circuitry 34 acquires the contrast-enhanced images g0 to g2 from the memory 33.

As described above, according to the seventh embodiment, the processing circuitry 34 in the X-ray diagnostic apparatus 10 acquires zeroth contrast-enhanced image g0 when the heart of the subject is in the reference state, the first contrast-enhanced image g1 when the heart is in the acetylcholine stress state, and the second contrast-enhanced image g2 when the heart is in the adenosine stress state. The processing circuitry 34 calculates the first blood flow ratio representing the ratio between the zeroth blood flow rate K1 in the myocardium in the reference state and the first blood flow rate K1_ACh in the myocardium in the acetylcholine stress state based on the zeroth contrast-enhanced image g0 and the second contrast-enhanced image g2. The processing circuitry 34 calculates the second blood flow ratio representing the ratio between the zeroth blood flow rate K1 and the second blood flow rate K1_ATP in the myocardium in the adenosine stress state based on the zeroth contrast-enhanced image g0 and the second contrast-enhanced image g2. The processing circuitry 34 causes the display 32 to display the first and second blood flow ratios side by side. The arrangement in which the processing circuitry 34 in the X-ray diagnostic apparatus 10 calculates blood flow ratios from contrast-enhanced images in this manner can obtain the functions and effects of each embodiment described above in real time after contrast medium radiography.

According to at least one of the embodiments described above, in an INOCA examination, blood flow can be quantitatively measured without using any sensor wire.

The term “processor” used in the above description means a circuit such as a CPU (Central Processing Unit), GPU (Graphics Processing Unit), ASIC(Application Specific Integrated Circuit), SPLD (Simple Programmable Logic Device), CPLD (Complex Programmable Logic Device), or FPGA (Field Programmable Logic Device). When the processor is, for example, a CPU, the processor reads out a program stored in a storage circuit and executes the program to implement the corresponding function. In contrast to this, if the processor is, for example, an ASIC, the corresponding function is directly incorporated as a logic circuit in the circuit of the processor instead of saving a program in the storage circuit. Note that each processor according to this embodiment may be formed as one processor by combining a plurality of independent circuits to implement the corresponding function as well as being formed as a single circuit. In addition, a plurality of constituent elements in FIGS. 1, 13, and 16 may be integrated into one processor to implement the corresponding function.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A medical image processing apparatus comprising processing circuitry configured to
acquire a zeroth contrast-enhanced image when a heart of a subject is in a reference state, a first contrast-enhanced image when the heart is in an acetylcholine stress state, and a second contrast-enhanced image when the heart is in an adenosine stress state,
calculate a first blood flow ratio representing a ratio between a zeroth blood flow rate in a myocardium in the reference state and a first blood flow rate in the acetylcholine stress state based on the zeroth contrast-enhanced image and the first contrast-enhanced image,
calculate a second blood flow ratio representing a ratio between the zeroth blood flow rate and a second blood flow rate in the myocardium in the adenosine stress state based on the zeroth contrast-enhanced image and the second contrast-enhanced image, and
cause a display to display the first blood flow ratio and the second blood flow ratio side by side.

2. The apparatus according to claim 1, wherein the processing circuitry is configured to cause the display to display the first blood flow ratio and the second blood flow ratio in at least one of a numerical value form and an image form.

3. The apparatus according to claim 2, wherein the processing circuitry is configured to
set a region of interest common to the zeroth contrast-enhanced image, the first contrast-enhanced image, and the second contrast-enhanced image,
calculate the first blood flow ratio in each of the interest regions in the zeroth contrast-enhanced image and the first contrast-enhanced image,
calculate the second blood flow ratio in each of the interest of regions in the zeroth contrast-enhanced image and the second contrast-enhanced image, and
when displaying in the numerical value form, display the first blood flow ratio in the first display form if the first blood flow ratio in the interest of region is not more than a first threshold, and display the second blood flow ratio in the second display form if the second blood flow ratio in the region of interest is not more than a second threshold.

4. The apparatus according to claim 3, wherein the first display form is a form of performing color display or alert display to indicate abnormality in the first blood flow ratio, and
the second display form is a form of performing color display or alert display to indicate abnormality in the second blood flow ratio.

5. The apparatus according claim 2, wherein the processing circuitry is configured to calculate the first blood flow ratio for each corresponding pixel in the zeroth contrast-enhanced image and the first contrast-enhanced image,
calculate the second blood flow ratio for each corresponding pixel in the zeroth contrast-enhanced image and the second contrast-enhanced image,
generate a first ratio image having a pixel value corresponding to the first blood flow ratio calculated for the each pixel and a second ratio image having a pixel value corresponding to the second blood flow ratio calculated for the each pixel, and
when displaying in the image form, cause the display to perform color display of the first ratio image and the second ratio image by assigning a color to the pixel value.

6. The apparatus according to claim 5, wherein the processing circuitry is configured to assign a color to the pixel value so as to align a baseline of a display range of the first ratio image with a baseline of a display range of the second ratio image.

7. The apparatus according to claim 5, wherein the processing circuitry is configured to perform color display of the first ratio image and the second ratio image so as to assign the same color to a pixel value corresponding to a first threshold for discriminating the first blood flow ratios into a normal value and an abnormal value in the first ratio image and a pixel value corresponding to a second threshold for discriminating the second blood flow ratios into a normal value and an abnormal value in the second ratio image.

8. The apparatus according to claim 1, wherein the processing circuitry is configured to
set a region of interest common to the zeroth contrast-enhanced image, the first contrast-enhanced image, and the second contrast-enhanced image,
calculate the first blood flow ratio in the region of interest in each of the zeroth contrast-enhanced image and first contrast-enhanced image,
calculate the second blood flow ratio in the region of interest in each of the zeroth contrast-enhanced image and the second contrast-enhanced image, and
display, side by side, a first graph representing the first blood flow ratio and a first threshold in the region of interest and a second graph representing the second blood flow ratio and a second threshold in the region of interest.

9. The apparatus according to claim 8, wherein the processing circuitry is configured to display the first graph and the second graph side by side so as to align a baseline of a description range of the first graph with a baseline of a description range of the second graph.

10. The apparatus according to claim 9, wherein the processing circuitry is configured to display the first graph and the second graph side by side so as to align the first threshold in the first graph with the second threshold in the second graph.

11. The apparatus according to claim 10, wherein the processing circuitry is configured to display a normal range and an abnormal range in the first graph, with the first threshold being a boundary, and display a normal range and an abnormal range in the second graph, with the second threshold being a boundary.

12. The apparatus according to claim 1, wherein the processing circuitry is configured to
set a region of interest common to the zeroth contrast-enhanced image, the first contrast-enhanced image, and the second contrast-enhanced image,
calculate the first blood flow ratio in the region of interest of each of the zeroth contrast-enhanced image and the first contrast-enhanced image,
calculate the second blood flow ratio in the region of interest in each of the zeroth contrast-enhanced image and the second contrast-enhanced image, and
cause the display to display a comparison graph comparatively showing the first blood flow ratio and the second blood flow ratio in the region of interest with respect to the reference value if a blood flow ratio at the zeroth blood flow rate is 1 as a reference value.

13. The apparatus according to claim 12, wherein the processing circuitry is configured to display a normal range and an abnormal range in the comparison graph with respect to each of the first blood flow ratio and the second blood flow ratio.

14. The apparatus according to claim 12, wherein the processing circuitry is configured to display the first blood flow ratio in the comparison graph while a description range is inverted centered on the reference value and display a threshold for each of the first blood flow ratio and the second blood flow ratio in the comparison graph.

15. The apparatus according to claim 1, wherein the processing circuitry is further configured to
acquire a myocardial blood flow image when the heart is in the acetylcholine stress state,
determine, based on the myocardial blood flow image, whether a myocardial blood flow in the subject has decreased, determine, based on the first contrast-enhanced image, whether a blood vessel in the subject is coarctated, detect coronary spasm upon determining that the myocardial blood flow has decreased and the blood vessel is coarctated, and detect microvascular spasm upon determining that the myocardial blood flow has decreased and the blood vessel is not coarctated, and
cause the display to display a detection result.

16. The apparatus according to claim 1, wherein the processing circuitry is configured to
calculate the first blood flow ratio by dividing pixel values of corresponding pixels in myocardial region in the zeroth contrast-enhanced image and myocardial region in the first contrast-enhanced image, and
calculate the second blood flow ratio by dividing pixel values of corresponding pixels in myocardial region in the zeroth contrast-enhanced image and myocardial region in the second contrast-enhanced image.

17. The apparatus according to claim 1, wherein the processing circuitry is configured to
calculate a third blood flow ratio by dividing pixel values of corresponding pixels in the myocardial region in the first contrast-enhanced image and the myocardial region in the second contrast-enhanced image, and
cause the display to further display the third blood flow ratio.

18. The apparatus according to claim 1, wherein the processing circuitry is configured to cause the display to further display at least the first blood flow rate and the second blood flow rate of the zeroth blood flow rate, the first blood flow rate, and the second blood flow rate side by side.

19. The apparatus according to claim 1, wherein the zeroth contrast-enhanced image, the first contrast-enhanced image, and the second contrast-enhanced image are medical images obtained by radiography under substantially the same radiography conditions except for a state of the heart.

20. An X-ray diagnostic apparatus comprising a processing circuitry configured to
acquire a zeroth contrast-enhanced image when a heart of a subject is in a reference state, a first contrast-enhanced image when the heart is in an acetylcholine stress state, and a second contrast-enhanced image when the heart is in an adenosine stress state,
calculate a first blood flow ratio representing a ratio between a zeroth blood flow rate in a myocardium in the reference state, and a first blood flow rate in the myocardium in the acetylcholine stress state based on the zeroth contrast-enhanced image and the first contrast-enhanced image,
calculate a second blood flow ratio representing a ratio between the zeroth blood flow rate and a second blood flow rate in the myocardium in the adenosine stress state based on the zeroth contrast-enhanced image and the second contrast-enhanced image, and
cause a display to display the first blood flow ratio and the second blood flow ratio side by side.
